(19) United States
(12) Patent Application Publication
(10) Pub. No.: US 2010/0330050 A1
(43) Pub. Date: Dec. 30, 2010

(54) USE OF HUMAN STEM CELLS AND/OR FACTORS THEY PRODUCE TO PROMOTE ADULT MAMMALIAN CARDIAC REPAIR THROUGH CARDIOMYOCYTE CELL DIVISION

(76) Inventors: Sergey V. Doronin, Stony Brook, NY (US); Glenn Gaudette, Holden, MA (US); Richard B. Robinson, Cresskill, NJ (US); Michael R. Rosen, New York, NY (US); Ira S. Cohen, Stony Brook, NY (US); Peter R. Brink, Setauket, NY (US)

Correspondence Address: KENYON & KENYON LLP, ONE BROADWAY, NEW YORK, NY, 10004, US

(21) Appl. No.: 12/781,498

(22) Filed: May 17, 2010

Related U.S. Application Data
(63) Continuation of application No. 11/296,018, filed on Dec. 6, 2005, now abandoned.
Application No. 11/296,018 is a continuation-in-part of application No. 11/240,948, filed on Sep. 29, 2005.
Application No. 11/240,948 is a continuation-in-part of application No. PCT/US2005/035030, filed on Sep. 29, 2005.
(60) Provisional application No. 60/614,856, filed on Sep. 30, 2004.

(51) Int. Cl. A61K 35/12; A61K 38/02; A61K 38/46; A61K 38/18; A61K 35/00; A61P 9/00

(52) U.S. Cl. 424/93.21; 514/16.4; 424/94.67; 514/8.2; 514/8.6; 514/8.3; 424/93.1

(57) ABSTRACT

The present invention relates to methods and compositions for stimulating the proliferation of cardiomyocytes for enhancement of cardiac repair. The invention is based on the discovery that upon contact with stem cells, or conditioned media derived from said stem cells, terminally differentiated cardiomyocytes can be stimulated to enter the cell cycle. Additionally, scaffolds capable of attracting stem cells to the area of implantation have been shown to induce cardiomyocyte proliferation. The present invention further relates to the discovery that the Wnt-5A ligand, which binds to the frizzled receptor (fz), functions to stimulate cardiomyocyte proliferation. The methods and compositions of the invention may be used in the treatment of cardiac disorders including, but not limited to, myocardial dysfunction or infarction. The invention further relates to screening assays designed to identify compounds that modulate the proliferative activity of cardiomyocytes and the use of such compounds in the treatment of cardiac disorders.

This research was supported by USPHS-NHLBI grants HL20558, HL-28958 and HL-67101. The United States Government may have rights in this invention.

### 1. INTRODUCTION

The present invention relates to methods and compositions for stimulating the proliferation of cardiomyocytes for enhancement of cardiac repair. The invention is based on the discovery that upon contact with stem cells, or conditioned media derived from said stem cells, terminally differentiated cardiomyocytes can be stimulated to enter the cell cycle. Additionally, scaffolds capable of attracting stem cells to the area of implantation have been shown to induce cardiomyocyte proliferation. The present invention further relates to the discovery that the Wnt-5A ligand, which binds to the frizzled receptor (fz), functions to stimulate cardiomyocyte proliferation. The methods and compositions of the invention may be used in the treatment of cardiac disorders including, but not limited to, myocardial dysfunction or infarction. The invention further relates to screening assays designed to identify compounds that modulate the proliferative activity of cardiomyocytes and the use of such compounds in the treatment of cardiac disorders.

### 2. BACKGROUND OF INVENTION

Heart failure is a notoriously progressive disease, despite medical management. The increasing gap between the incidence of end-stage heart failure and surgical treatment is due, in great part, to the shortage of donor organs. Thus, there is a need for alternative approaches for treatment of damaged heart tissue that is not dependent of the availability of donor organs.

Following myocardial infarction, the heart does not reconstitute lost cardiomyocytes and the damaged tissue is eventually replaced by scar. This, however, does not rule out that regeneration of mammalian heart might occur under circumstances different from those of infarcted heart. For instance, zebrafish or amphibians reconstitute amputated parts of the heart and, in amphibians, heart regeneration occurs as a result of mitotic expansion of cardiomyocytes (Poss, K. D., Wilson, L. G. & Keating, M. T., 2002 Science 298, 2188-2190; Rumyantsev, P. P., 1973, Z. Zellforsch. Mikrosk. Anat. 139, 431-450; Flink, I. L., 2002, Anat. Embryol. (Berl) 205, 235-244; and Borisov, A. B., 1998, Cellular and molecular basis of regeneration from invertebrates to humans. Wiley, N.Y.).

A major therapeutic goal of modern cardiology is to design strategies aimed at minimizing myocardial necrosis and optimizing cardiac repair following myocardial infarction. The present invention provides novel methods and compositions for stimulating the proliferation of cardiomyocytes and the use of such methods and compositions for promotion of cardiac repair.

### 3. SUMMARY OF THE INVENTION

The present invention provides methods and compositions for stimulating proliferation of cardiomyocytes for enhancement of cardiac repair. The methods and compositions of the invention may be used in the treatment of cardiac disorders including, but not limited to, myocardial dysfunction and infarction. The invention further relates to screening assays designed to identify compounds that modulate the proliferative activity of cardiomyocytes and the use of such compounds in the treatment of cardiac disorders.

The invention is based on the discovery that upon contact with stem cells, or conditioned media derived from said stem cells, cardiomyocytes can be stimulated to enter the cell cycle. Specifically, when a full thickness portion of the canine right ventricle was replaced with a material made of natural extracellular scaffold, myocardium was partially regenerated eight weeks later that produced significant regional mechanical work. This regeneration was accompanied by propagation of c-kit positive stem cells in the implant at early stages of the regeneration process, and was later associated with a mitotically expanding population of cardiomyocytes. Additionally, this process was reconstituted in vitro by co-culturing cardiomyocytes with human mesenchymal stem cells, or treating cardiomyocytes with conditioned media derived from the human mesenchymal stem cells, and observing cardiomyocyte proliferation.

Further, the present invention is based on the discovery that the human Wnt-5 ligand, which is produced by stem cells, is capable of stimulating cardiomyocyte proliferation. The Wnt-5A receptors termed frizzleds, including human frizzled-2 (also termed as in early publications as hFz5), are expressed on the surface of cardiomyocytes.

Thus, the present invention is also based on the observation that given the proper environment, the mammalian heart can regenerate lost myocardium.

Accordingly, the present invention relates to a method for stimulating cardiomyocytes to enter the cell cycle comprising co-culturing stem cells and cardiomyocytes. In yet another embodiment of the invention, cardiomyocyte proliferation may be stimulated utilizing a method comprising contacting cardiomyocytes with stem cell conditioned media.

The present invention also provides a method for regenerating myocardium in a mammal comprising administering stem cells to the myocardium in a quantity sufficient to induce native cardiomyocytes to enter the cell cycle. Specifically, the invention relates to the use of stem cells to promote an increase in the number of cells in the myocardium through increased proliferation of native cardiac progenitor cells resident in the myocardium; stimulation of myocyte proliferation; and stimulation of differentiation of host cardiac progenitor stem cells into cardiac cells, for example. Such an increase in cell number results predominantly from stimulation of the native myocardium cells by factors produced by the administered stem cells.

In yet another embodiment of the invention, compositions capable of attracting native or endogenous stem cells to the myocardium may be administered to the myocardium. Such compositions include, but are not limited to, scaffolds that are capable of attracting stem cells the region of the myocardium in need of repair. Accordingly, the invention provides a method of effecting delivery of stem cells to an afflicted area of a heart, comprising administration of scaffolds to the region of the myocardium in need of repair, thereby attracting stem cells to the afflicted area of the myocardium.

The invention further relates to a method for treating a subject afflicted with a cardiac disorder, in vivo, comprising (i) producing a solution comprising media conditioned from the culture of stem cells, in vitro, and (ii) administering the solution of step (i) to the subject, thereby treating the cardiac disorder in the subject.

According to another aspect of the invention, a method for treating a subject afflicted with a cardiac disorder, in vivo, is provided, comprising (i) producing a solution comprising media conditioned from the co-culturing, in vitro, of stem cells and myocytes and (ii) administering the solution of step (i) to the subject, thereby treating the cardiac disorder in the subject.

In another embodiment of the invention, the administered scaffolds may be engineered to contain exogenously added stem cells which are capable of stimulating cardiomyocyte proliferation. Alternatively, the scaffold may be placed in contact with the conditioned medium derived from said stem cells as a means for delivery of biologically active components present in the medium which are capable of stimulating cardiomyocyte proliferation.

The present invention also provides methods for regenerating myocardium in a mammal comprising administration of compounds capable of modulating the Wnt-5A signal transduction pathways. Such compounds include those capable of activating the frizzled receptors including, but not limited to, the Wnt-5A protein. In a specific embodiment of the invention, modulators of the Wnt-5A signal transduction pathways, or pharmaceutical compositions containing such modulators, may be administered to the region of the myocardium in need of repair. Such compositions include, but are not limited to, Wnt-5A protein and/or scaffolds containing Wnt-5A protein. Such scaffolds serve as a means for delivery of sustained concentrations of Wnt-5A to the region of the myocardium in need of repair. Alternatively, cells know to express Wnt-5A, or cells genetically engineered to express Wnt-5A, may be administered alone or embedded within a scaffold.

In yet another embodiment, the present invention provides methods for identification of biologically active agents capable of modulating the proliferation of cardiomyocytes. Such agents include those produced by stem cells which have the potential to induce cardiomyocyte proliferation and thereby promote myocardium repair. Such biologically active agents include, but are not limited to, those capable of modulating the Wnt-5A signal transduction pathways. Such agents can be used to treat subjects suffering from cardiac disorders including, but not limited to, myocardial dysfunction or infarcation.

### 4. BRIEF DESCRIPTION OF THE FIGURES

FIG. 1. Cardiac regeneration with extracellular matrix (ECM) scaffold. FIG. 1A shows regional stroke work of the myocardium distant from the site of surgery (Baseline), eight week old ECM implant region (ECM, *=p<0.05 from Baseline) and Dacron implant region (Dacron, #=p<0.05 from ECM). FIG. 1B shows staining of the eight weeks old ECM implant region for α-sarcomeric actinin (a cardiomyocyte marker; FITC, green). Nuclei were counterstained with DAPI (blue). Schematic location of implant -myocardium border (dashed green line), the border of regenerated myocardium (solid red line), area A of the adjacent host myocardium, area B of the internal part of the implant, area C of the tip of the regeneration cone and non regenerated area D of the implant overlaid on haematoxylin and eosin staining of eight week old ECM implant region.

FIG. 2. Stem cells accumulation in Dacron and ECM implants. Two weeks old Dacron and ECM implants were stained for α-sarcomeric actinin (TRITC, red) and c-kit. (FITC, green). Nuclei were counterstained with DAPI (blue). FIG. 2A shows the border area of the Dacron implant with the host myocardium. FIG. 2B shows the border area of the ECM implant with the host myocardium. FIG. 2C shows internal area of ECM implant.

FIG. 3. Expression of cell division markers cyclin D1, Ki-67 and Wnt-5A in regenerated myocardium. Eight week old ECM implants were stained for cyclin D1 and Ki-67. Cardiomyocytes were visualized by staining for α-sarcomeric actinin (green). Nuclei were counterstained with DAPI (blue). FIG. 3A, shows cyclin D1 staining (red) of the epicardial surface of the implant. The layer of cyclin D1 positive cells is located under the surface layer of cyclin D1 negative cells. FIG. 3B shows the endocardial side of the implant with cardiomyocytes positive for cyclin D1. FIG. 3C shows nuclear localization of Ki-67 (red) in cardiomyocytes in the regenerating area. FIG. 3D shows the epicardial surface of the implant stained for Wnt-5A (green) and Ki-67 (red). Cell in the epicardial surface are Wint-5A positive and located above the layer of Ki-67 positive cells. FIG. 3E shows the endocardial side of the implant stained for cyclin D1 (red) and Wnt-5A (green). The endocardial surface of the implant is composed of Wnt-5A positive cells (blue arrow). Cyclin D1 positive cardiomyocytes (white arrow) are located above the Wnt-5A positive cell layer.

FIG. 4. Effects of human mesenchymal stem cells on cardiomyocytes in cell culture. Cardiomyocytes from canine hearts were co-cultured with human mesenchymal stem cells for 3-30 days in OMEN containing 5% of fetal bovine serum. Cells were labeled with BrdU and stained for cyclin D1 and Ki-67. Cardiomyocytes were visualized by staining for α-sarcomeric actinin. Nuclei and mitotic chromosomes were counterstained with DAPI (blue). FIG. 4A shows cardiomyocytes maintained in the absence of hMSCs for three days and stained for cyclin D1 (red) and α-sarcomeric actinin (green). FIG. 4B shows cyclin D1 expression (red) that was induced in cardiomyocytes after three days of co-culturing with hMSCs. Cardiomyocytes after five days in cell culture with hMSCs are shown on FIGS. 4C and D. FIG. 4C shows a myocyte in anaphase stained for cyclin D1 (red) and α-sarcomeric actinin (green). FIG. 4D shows Ki-67 positive cardiomyocytes in the intermediate phase of the cell cycle (yellow arrow) and mitotically inactive (Ki-67 negative) cardiomyocytes (white arrow). FIG. 4E shows a ten day old colony of cardiomyocytes labeled with BrdU (green). FIG. 4F shows two week old colonies of cardiomyocytes (white arrows) which are often interconnected by spontaneously contracting myocytes (blue arrow). FIG. 4G shows a thirty day old colony of cardiomyocytes that were viable and cyclin D1 positive. FIG. 4H shows two fourteen day old colonies of cardiomyocytes. Cardiomyocytes were stimulated to proliferate by media conditioned by human mesenchymal stem cells but were not cocultured with the stem cells. Cardiomyocyte colonies (white arrows) formed on the surface of cardiac fibroblasts which were also present in the initial myocyte preparation. The yellow arrow in the insert points to a mitotically silent cardiomyocyte.

FIG. 5A-D. FIG. 5A. Myocytes were found in the ECM implant region at eight weeks. FIG. 5B. Cyclin D1 expression was detected in the ECM implant region at eight weeks post-implantation. FIG. 5C. Nuclear expression of Ki-67 was observed in a myocyte from the regenerating region of the ECM patch at 8 weeks. FIG. 5D. Expression of CyclinD1 was observed in a myocyte from the regenerating region of the ECM patch at 8 weeks.

FIG. 6A-B. Ability of Scaffolds to Stimulate Cardiac Regeneration. FIG. 6A. Implants were done with Dacron, ECM scaffold and Veritas® scaffold. Workloops are shown from the implant region eight weeks post-implantation. FIG. 6B. Comparison of regional work at eight weeks with ECM, Dacron and Veritas® demonstrates that over the eight week implantation period, the ECM and Veritas® scaffold became a contractile tissue.

FIG. 7A-B. Veritas® Scaffold Allows for Myocardial Regeneration. FIG. 7A demonstrates the presence of myocytes within the Veritas® patch implant region. FIG. 7B demonstrates staining of the α-sarcomeric actinin, a marker for cardiac myocytes.

FIG. 8A-B. Wnt-5A Expression during Myocardium Regeneration. FIG. 8A. Wnt-5A is expressed at 8 weeks post-implantation in regenerating myocardium. FIG. 8B. Wnt-5A co-localizes with newly formed blood vessels.

FIG. 9. Overexpression of Wnt-5A in recombinantly engineered human mesenchymal stem cells. Western Blots demonstrate the expression of Wnt-5A and HA-tagged Wnt-5A in transfected human mesenchymal stem cells.

FIG. 10. Stimulation of cyclin D1 expression in the nucleus of myocytes by media conditioned with hMSCs overexpressing Wnt-5A was demonstrated after 4 days in cell culture.

### 5. DETAILED DESCRIPTION OF THE INVENTION

Described herein is the discovery that stem cells are capable of stimulating cardiomyocytes to enter the cell cycle. Thus, the present invention relates to methods and compositions for stimulating the proliferation of cardiomyocytes for enhancement of cardiac repair. The methods and compositions of the invention may be used in the treatment of cardiac disorders including, but not limited to, myocardial dysfunction or infarction. The invention further relates to screening assays designed to identify compounds that modulate the proliferation of cardiomyocytes and the use of such compounds in the treatment of cardiac disorders. The invention is described in detail in the subsections below.

5.1. Stem Cells and Conditioned Medium

The present invention encompasses methods for regenerating myocardium in a mammal comprising administering stem cells to the myocardium in a quantity sufficient to induce native cardiomyocytes to enter the cell cycle. Specifically, the invention relates to the use of stem cells to promote an increase in the number of cells in the myocardium through increased proliferation of native cardiac progenitor cells resident in the myocardium; stimulation of myocyte proliferation; and/or stimulation of differentiation of host cardiac progenitor stem cells into cardiac cells, for example. Such an increase in cell number results predominantly from stimulation of the native myocardium cells by factors produced by the administered stem cells.

As used herein, “stem cell” refers to any cell having the potential to differentiate into one or more different cell types. Such cells include, but are not limited to, stem cells derived from a variety of different sources including, for example, bone marrow, embryonic blastocysts or yolk sac, spleen, blood, including peripheral blood and umbilical cord blood, adipose tissue and other tissues and organs. Such stem cells include, but are not limited to, hematopoietic stem cells, endothelial stem cells or embryonic stem cells. In a preferred embodiment of the invention, mammalian mesenchymal stem cells are utilized in the practice of the invention. In a preferred embodiment of the invention the utilized stem cells are derived from a human.

Stem cells may be obtained from a variety of different donor sources. In a preferred embodiment, autologous stem cells are obtained from the subject who is to receive the transplanted stem cells to avoid immunological rejection of foreign tissue. In yet another preferred embodiment of the invention, allogenic stem cells may be obtained from donors who are genetically related to the recipient and share the same transplantation antigens on the surface of their stem cells. Alternatively, stem cells may be derived from antigenically matched (identified through a national registry) donors. In instances where antigenically matched stem cells cannot be located, non-matched cells may be used, however, it may be necessary to administer immunosuppressive agents to prevent recipient rejection of the donor stem cells.

Procedures for harvest and isolation of such stem cells are well known to those of skill in the art and do not differ from those used in conventional stem cell transplantation. Adult stem cells may be derived from bone marrow, peripheral blood, adipose tissue and other adult tissues and organs. For derivation of embryonic stem cells, stem cells can be extracted from the embryonic inner cell mass during the blastocyst stage. Fetal stem cells may be derived from the liver, spleen, brain or heart of fetuses, 4-12 weeks gestation, following elective abortions, terminated ectopic pregnancies or spontaneous miscarriages. In a preferred embodiment of the invention, mesenchymal stem cells are derived from adult bone marrow.

In a non-limiting embodiment of the invention, antibodies that bind to cell surface markers selectively expressed on the surface of stem cells may be used to identify or enrich for populations of stem cells using a variety of methods. Such markers include, for example, CD34, SSEA3, SSEA4, anti-TRA1-60, anti-TRA1-81 or c-kit.

Prior to administration of stem cells, the cells may be genetically engineered to express proteins that further enhance the ability of such cells to enhance cardiomyocyte proliferation. For example, in a non-limiting embodiment, the cells may be engineered to over express the Wnt-5A protein and/or insulin-like growth factor-1.

The invention also encompasses methods wherein culture media conditioned by stem cells is used to induce cardiomyocyte proliferation. According to another aspect of the invention, a method for treating a subject afflicted with a cardiac disorder, in vivo, is provided, comprising (i) producing a solution capable of inducing myocyte proliferation and (ii) administering the solution of step (i) to the subject, thereby treating the cardiac disorder in the subject.

In a further embodiment of the invention, media conditioned by myocytes and stem cells when they are co-cultured together is used to stimulate cardiomyoctye proliferation. According to another aspect of the invention, a method for treating a subject afflicted with a cardiac disorder, in vivo, is provided, comprising (i) producing a solution comprising media conditioned from the culture of cells, in vitro, and (ii) administering the solution of step (i) to the subject, thereby treating the cardiac disorder in the subject.

The invention also encompasses the use of such conditioned media wherein the media has been processed to increase the concentration of the biologically active components of the media, i.e., those capable of inducing cardiomyocyte proliferation. To this end, the conditioned media may be placed in an environment wherein the solution undergoes ultrafiltration or lyophilization leading to a more concentrated media solution.

Prior to the use of the conditioned media, additional components may be added to enhance the ability of the media to induce cardiomyocyte proliferation. For example, in non-limiting embodiments of the invention, the solution may further comprise Wnt-5A protein, metalloproteases (MMPs), insulin-like growth factor, platelet derived growth factor, and/or brain derived neurotrophic factor, secreted frizzled-related protein(s) and dickkopf-1 (DKK1).

The present invention provides methods for identification of the biologically active components of the conditioned media capable of stimulating cardiomyocyte proliferation. For example, methods well known to those of skill in the art may be used to enrich, or purify to homogeneity, said components from the conditioned media. Such methods include, but are not limited to chromatographic methods that can be used to enrich or purify components based on the overall charge or size of the component. A variety of different assays may be used to identify the fractions containing the component of interest, including but not limited to methods of measuring cardiomyocyte proliferation or changes in levels, or modification, of proteins known to be associated with cell proliferation. Such assays are described in further detail below.

Alternatively, cloning methods well known to those of skill in the art may be used to isolate a nucleic acid sequence encoding a component of the conditioned media capable of stimulating cardiomyocyte proliferation. For example, a cDNA library may be constructed utilizing mRNA derived from stem cells known to express the component of interest. The resulting cDNA library may then be introduced into a cell line that does not normally express the component of interest and the conditioned media from the transfected cells can be assayed for its ability to promote cardiomyocyte proliferation.

A variety of different assays may be used to identify cells expressing the component of interest, including but not limited to methods of measuring cardiomyocyte proliferation or changes in levels, or modification, of proteins known to be associated with cell proliferation. Accordingly, the present invention relates to an assay for identifying the presence of a component that stimulates myocyte proliferation comprising: (i) co-culturing, in vitro, cells and myocytes, in which the component is expressed in the cells; (ii) measuring the amount of myocyte cell division after step (i); (iii) repeating step (i) in the presence of cells either not expressing the component or having low level of expression of the component; (iv) measuring the amount of myocyte cell division after step (iii); and (v) comparing the measurements of step (ii) and step (iv), whereby the amount of myocyte cell division as measured in step (ii) being greater than the amount of myocyte cell division as measured in step (iv) indicates the presence of a component that stimulates myocyte proliferation.

Through multiple rounds of purification of cDNAs from positive pools of cells, subsequent transfections and assaying of the conditioned media, a single cell clone containing a cDNA encoding a component of interest may be obtained.

5.2. Wnt-5A Stimulates Cardiomycyte Proliferation

The WNT gene family consists of structurally related genes which encode secreted signaling proteins. These proteins have been implicated in oncogenesis and in several developmental processes, including regulation of cell fate and patterning during embryogenesis. The human Wnt-5 gene is a member of the WNT gene family which encodes a protein which shows 98%, 98% and 87% amino acid identity to the mouse, rat and the xenopus Wnt-5A protein, respectively. The frizzled receptors, including frizzled-2, frizzled-3, frizzled-4, frizzled-5, frizzled-6, and frizzled-8 have been demonstrated to be the receptors for the Wnt-5A protein (Takada, R., Hijikata, H., Kondoh, H., Takada, S. Genes to Cells, 2005, 10, 919-928). The sequence of the frizzled-2 gene, termed below as hFz5 according to the initial classification, is disclosed in Gene Bank ID: 3927885. Embodiments of the invention are described below for human Wnt-5A and hFz5, however, it is understood that other Wnt proteins and frizzled receptors derived from species other than human may be used equally well due to redundancy of Wnt signaling pathways and lack of cross-species specificity.

As described herein it has been discovered that the Wnt-5A protein produced by stem cells is capable of stimulating cardiomyocyte proliferation. Accordingly, the present invention relates to methods for stimulating cardiomyocyte proliferation comprising modulation of the Wnt-5A signal transduction signal transduction pathways. In an embodiment of the invention the Wnt-5A signal transduction pathways are activated. In yet another embodiment of the invention, a fizzled ligand is utilized to stimulate cardiomyocyte proliferation. In a non-limiting embodiment of the invention, the Wnt-5A ligand is used to stimulate cardiomyocyte proliferation.

In yet another embodiment of the invention the Wnt-5A signal transduction pathway is inhibited. Inhibitors of Wnt-signaling include, but are not limited to, secreted frizzled-related protein(s) and Dickkopf-1 (DKK1). Due to the differential effect of Wnt-signaling on cellular proliferation, it may be desirable in some cases to inhibit Wnt-signaling to control the cellular environment. For example, in some instances proliferation of fibroblasts may be undesirable and inhibited to prevent excessive collagen deposition.

Additionally, it may be desirable to stimulate cell proliferation at certain specific times using activators of the Wnt-signaling pathway, while at other times it may be desirable to inhibit cell proliferation using inhibitors of the Wnt-signaling pathway. For example, to permit differentiation of stem cells into cardiomyocytes the cells should be permitted to go through a complete cell cycle. Therefore, cell proliferation may be stimulated initially to induce multiplication of native stem cells and promote vascularization. At later times, the cell cycle may be inhibited to promote stem cell differentiation and, possibly, to inhibit propagation of cardiac fibroblasts.

The present invention also relates to a method for treating a subject afflicted with a cardiac disorder comprising administering a protein capable of activating the hFz5 receptor in an amount sufficient to stimulate the in vivo proliferation of cardiomyocytes and thereby promote myocardium repair. In a non-limiting embodiment of the invention, the Wnt-5A ligand is administered to promote myocardium repair.

In addition to full length Wnt-5A protein, peptide fragments that retain the ability to stimulate cardiomyocyte proliferation may be utilized for stimulation of myocardium repair. Identification of Wnt-5A peptide fragments that retain biological activity, i.e., induction of cardiomyocyte proliferation, may be accomplished using methods well known to those of skill in the art. For example, Wnt-5A peptide fragments may be recombinantly expressed and tested directly to determine whether they are capable of stimulating cardiomyocyte entry into the cell cycle. Alternatively, conditioned media derived from cells engineered to express Wnt-5A peptide fragments may be tested for its ability to induce cell proliferation. In a preferred embodiment, when conditioned media is to be tested, the engineered cell is one that normally does not express Wnt-5A or expresses low levels thereof.

Methods of measuring cell proliferation are well known in the art and most commonly include determining DNA synthesis characteristic of cell replication. There are numerous methods in the art for measuring DNA synthesis, any of which may be used according to the invention. For example, DNA synthesis may be determined using a radioactive label ([³H]-thymidine) or the nucleoside analog BrdU for detection by immunofluorescence. Additionally, the cells may be assayed to determine whether there are changes in levels, or modification, of proteins known to be associated with cell proliferation. Such proteins include, for example, cyclin D1, CDK4, p107 or retinoblastoma protein. The efficacy of the Wnt-5A peptide fragments can be assessed by generating dose response curves from data obtained using various concentrations of the protein. A control assay can also be performed to provide a baseline for comparison. Identification of the cardiomyoctye proliferation amplified in response to a Wnt-5A peptide fragments can be carried out according to such phenotyping as described above.

Various delivery systems are known and can be used to transfer the Wnt-5A proteins of the invention the region of heart in need of repair, e.g., encapsulation in liposomes, microparticles, microcapsules, recombinant cells capable of expressing Wnt-5A, receptor-mediated endocytosis (see, e.g., Wu and Wu, 1987, J. Biol. Chem. 262:4429-4432), construction of a nucleic acid expressing Wnt-5A polypeptides as part of a retroviral, adenoviral, adeno-associated viral or other vector, injection of DNA, electroporation, calcium phosphate mediated transfection, etc.

To practice the methods of the invention it may be necessary to recombinantly express the Wnt-5A protein. The cDNA sequence and deduced amino acid sequence of Wnt-5A has been characterized from several species including human, mouse, rat and the xenopus. Sequences of the Wnt-proteins are available from public databases. The GenBank ID for human Wnt-5A is GI:731157. Cloned Wnt-5A and other proteins of Wnt-family are commercially available from various sources, for instance, from Upstate Biotechology Inc.

Wnt-5A nucleotide sequences may be isolated using a variety of different methods known to those skilled in the art. For example, a cDNA library constructed using RNA from a tissue known to express Wnt-5A can be screened using a labeled Wnt-5A probe. Alternatively, a genomic library may be screened to derive nucleic acid molecules encoding the Wnt-5A protein. Further, Wnt-5A nucleic acid sequences may be derived by performing a polymerase chain reaction (PCR) using two oligonucleotide primers designed on the basis of known Wnt-5A nucleotide sequences. The template for the reaction may be cDNA obtained by reverse transcription of mRNA prepared from cell lines or tissue known to express Wnt-5A.

Wnt-5A protein, polypeptides and peptide fragments, mutated, truncated or deleted forms of Wnt-5A and/or Wnt-5A fusion protein can be prepared for a variety of uses, including but not limited to the identification of other cellular gene products involved in the regulation of Wnt-5A mediated cardiomyocyte cell proliferation and the screening for compounds that can be used to modulate cardiomyocyte cell proliferation. Wnt-5A fusion proteins include fusions to an enzyme, fluorescent protein, a polypeptide tag or luminescent protein which provide a marker function.

While the Wnt-5A polypeptides and peptides can be chemically synthesized (e.g., see Creighton, 1983, Proteins: Structures and Molecular Principles, W. H. Freeman & Co., N.Y.), large polypeptides derived from Wnt-5A and the full length Wnt-5A itself may be advantageously produced by recombinant DNA technology using techniques well known in the art for expressing a nucleic acid containing Wnt-5A gene sequences and/or coding sequences. Such methods can be used to construct expression vectors containing the Wnt-5A nucleotide sequences and appropriate transcriptional and translational control signals. These methods include, for example, in vitro recombinant DNA techniques, synthetic techniques, and in vivo genetic recombination. (See, for example, the techniques described in Sambrook et al., 1989, supra, and Ausubel et al., 1989, supra).

A variety of host-expression vector systems maybe utilized to express the Wnt-5A nucleotide sequences. The expression systems that may be used for purposes of the invention include but are not limited to microorganisms such as bacteria transformed with recombinant bacteriophage, plasmid or cosmid DNA expression vectors containing Wnt-5A nucleotide sequences; yeast transformed with recombinant yeast expression vectors containing Wnt-5A nucleotide sequences or mammalian systems harboring recombinant expression constructs containing promoters derived from the genome of mammalian cells or from mammalian or viruses.

Appropriate expression systems can be chosen to ensure that the correct modification, processing and sub-cellular localization of the Wnt-5A protein occurs. To this end, eukaryotic host cells which possess the ability to properly modify and process the Wnt-5A protein are preferred. For long-term, high yield production of recombinant Wnt-5A protein, such as that desired for development of cell lines for screening purposes, stable expression is preferred. Rather than using expression vectors which contain origins of replication, host cells can be transformed with DNA controlled by appropriate expression control elements and a selectable marker gene, i.e., tk, hgprt, dhfr, neo, and hygro gene, to name a few. Following the introduction of the foreign DNA, engineered cells may be allowed to grow for 1-2 days in enriched media, and then switched to a selective media. Such engineered cell lines may be particularly useful in screening and evaluation of compounds that modulate the endogenous activity of the Wnt-5A gene product.

The compositions and methods of the invention can be used to provide sequences encoding a Wnt-5A protein to cells of an individual with a cardiac disorder. The compositions and methods of the invention can be used to induce proliferation of cardiomyocytes in an individual with a cardiac disorder, for example.

In a preferred embodiment, nucleic acids comprising a sequence encoding a Wnt-5A are administered to promote cardiomyocyte proliferation, by way of gene delivery and expression into a host cell. In this embodiment of the invention, the nucleic acid mediates an effect by promoting Wnt-5A production. Any of the methods for gene delivery into a host cell available in the art can be used according to the present invention. For general reviews of the methods of gene delivery see Strauss, M. and Barranger, J. A., 1997, Concepts in Gene Therapy, by Walter de Gruyter & Co., Berlin; Goldspiel et al., 1993, Clinical Pharmacy 12:488-505; Wu and Wu, 1991, Biotherapy 3:87-95; Tolstoshev, 1993, Ann. Rev. Pharmacol. Toxicol. 33:573-596; Mulligan, 1993, Science 260:926-932; and Morgan and Anderson, 1993, Ann. Rev. Biochem. 62:191-217; 1993, TIBTECH 11(5):155-215. Exemplary methods are described below.

Delivery of the nucleic acid molecule encoding Wnt-5A into a host cell may be either direct, in which case the host is directly exposed to the nucleic acid molecule, or indirect, in which case, host cells are first transformed with the Wnt-5A encoding nucleic acid molecule in vitro, and then transplanted into the host. These two approaches are known, respectively, as in vivo or ex vivo gene delivery.

In a specific embodiment, the nucleic acid is directly administered in vivo, where it is expressed to produce Wnt-5A. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., Wu and Wu, 1987, J. Biol. Chem. 262:4429-4432).

In a specific embodiment, a viral vector that contains Wnt-5 encoding nucleic acid sequences can be used. For example, a retroviral vector can be utilized that has been modified to delete retroviral sequences that are not necessary for packaging of the viral genome and integration into host cell DNA (see Miller et al., 1993, Meth. Enzymol. 217:581-599). Alternatively, adenoviral or adeno-associated viral vectors can be used for gene delivery to cells or tissues. (See, Kozarsky and Wilson, 1993, Current Opinion in Genetics and Development 3:499-503 for a review of adenovirus-based gene delivery).

In a preferred embodiment of the invention an adeno-associated viral vector may be used to deliver nucleic acid molecules capable of encoding Wnt-5A. The vector is designed so that, depending on the level of expression desired, the promoter and/or enhancer element of choice may be inserted into the vector.

Another approach to gene delivery into a cell involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. The resulting recombinant cells can be delivered to a host by various methods known in the art. In a preferred embodiment, the cell used for gene delivery is autologous to the host cell.

In a specific embodiment of the invention, cells may be removed from a subject having a cardiac disorder and transfected with a nucleic acid molecule capable of encoding Wnt-5A. Cells may be further selected, using routine methods known to those of skill in the art, for integration of the nucleic acid molecule into the genome thereby providing a stable cell line expressing the Wnt-5A protein. Such cells are then transplanted into the subject thereby providing a source of protein capable of promoting cardiac repair.

In addition to gene therapy methods, other methods may be used to deliver the Wnt-5A protein to the region of the heart in need of repair. Accordingly, the Wnt-5A may be administered to a subject afflicted with a cardiac disorder embedded or incorporated into a scaffold. The use of a scaffold provides a means for long term sustained release of Wnt-2A to the region of the myocardium in need of repair. A more detailed discussion of scaffolds that may be used in the practice of the invention is set forth below.

The present invention further provides screening assays for identification of compounds capable of promoting cardiac repair. In a preferred embodiment of the invention, the screening assays are designed to identify compounds capable of modulating the Wnt-5A signal transduction pathways. This aspect of the invention is based on the surprising discovery that the Wnt-5A protein is capable of stimulating myocardiocyte proliferation.

The present invention encompasses screening assays designed for the identification of modulators of the Wnt-5A signal transduction pathways. The invention further relates to the use of such modulators in the treatment of cardiac disorders based on the ability of Wnt-5A to induce cardiomyocyte proliferation.

In accordance with the invention, non-cell based assay systems may be used to identify compounds that interact with, i.e., bind to the Wnt-5A or hFz5 receptor, and regulate the proliferation of cardiomyocytes. Such compounds may be used to regulate cardiomyocyte proliferation.

Recombinant Wnt-5A, including peptides corresponding to different functional domains, or Wnt-5A fusion proteins, may be expressed and used in assays to identify compounds that interact with Wnt-5A. Alternatively, recombinant hFz5, including peptides corresponding to different functional domains or hFz5 fusion proteins may be expressed and used in assays to identify compounds that interact with the hFz5 protein.

To this end, soluble Wnt-5A or hFz5 maybe recombinantly expressed and utilized in non-cell based assays to identify compounds that bind to Wnt-5A or hFz5. Recombinantly expressed Wnt-5A or hFz5 polypeptides or fusion proteins containing one or more of the Wnt-5A or hFz5 functional domains may be prepared as described above, and used in the non-cell based screening assays. For example, the full length Fz protein, or a soluble truncated hFz5 protein, e.g., in which the one or more of the cytoplasmic and transmembrane domains is deleted from the molecule, a peptide corresponding to the extracellular domain, or a fusion protein containing the hFz5 extracellular domain fused to a protein or polypeptide that affords advantages in the assay system (e.g., labeling, isolation of the resulting complex, etc.) can be utilized.

The hFz5 protein may also be one which has been fully or partially isolated from cell membranes, or which may be present as part of a crude or semi-purified extract. As a non-limiting example, the hFz5 protein may be present in a preparation of cell membranes. In particular embodiments of the invention, such cell membranes may be prepared using methods known to those of skill in the art.

The principle of the assays used to identify compounds that bind to Wnt-5A or hFz5 involves preparing a reaction mixture of the Wnt-5A or hFz5 protein and the test compound under conditions and for time sufficient to allow the two components to interact and bind, thus forming a complex which can be removed and/or detected in the reaction mixture. The identity of the bound test compound is then determined.

The screening assays are accomplished by any of a variety of commonly known methods. For example, one method to conduct such an assay involves anchoring the Wnt-5A or hFz5 protein, polypeptide, peptide, fusion protein or the test substance onto a solid phase and detecting Wnt-5A/test compound or hFz5/test compound or Wnt-5A/test compound or hFz5/test compound complexes anchored on the solid phase at the end of the reaction. In one embodiment of such a method, the Wnt-5A or hFz5 reactant is anchored onto a solid surface, and the test compound, which is not anchored, may be labeled, either directly or indirectly.

In practice, microtitre plates conveniently can be utilized as the solid phase. The anchored component is immobilized by non-covalent or covalent attachments. The surfaces may be prepared in advance and stored. In order to conduct the assay, the non-immobilized component is added to the coated surfaces containing the anchored component. After the reaction is completed, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the solid surface; e.g., using a labeled antibody specific for the previously non-immobilized component.

Alternatively, a reaction is conducted in a liquid phase, the reaction products separated from unreacted components using an immobilized antibody specific for Wnt-5A or hFz5 protein, fusion protein or the test compound, and complexes detected using a labeled antibody specific for the other component of the possible complex to detect anchored complexes.

In another embodiment of the invention, computer modeling and searching technologies will permit identification of potential modulators of Wnt-5A signal transduction pathways. The three dimensional geometric structure of the active site may be determined using known methods, including x-ray crystallography, which can determine a complete molecular structure. On the other hand, solid or liquid phase NMR can be used to determine certain intramolecular distances. Any other experimental method of structure determination can be used to obtain the partial or complete geometric structure of the Wnt-5A or hFz5 active site.

Having determined the structure of the Wnt-5A or hFz5 active site, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential Wnt-5A or hFz5 modulating compounds.

In accordance with the invention, non-cell based assays are to be used to screen for compounds that directly inhibit or activate the Wnt-5A/hFz5 signal transduction pathway. Such activities include but are not limited to induction or inhibition of cardiomyocyte proliferation. Thus, in a preferred embodiment of the invention, any compounds identified using the non-cell based methods described above, are further tested to determine their ability to modulate cardiomyocyte proliferation.

In accordance with the invention, cell based assay systems can be used to screen for compounds that modulate the activity of the Wnt-5A signal transduction pathways. To this end, cells that endogenously express hFz5 can be used to screen for compounds. Such cells include, for example, cardiomyocytes. Alternatively, cell lines, such as 293 cells, COS cells, CHO cells, fibroblasts, and the like, genetically engineered to express Fz can be used for screening purposes.

In accordance with the invention, a cell-based assay system is provided that can be used to screen for compounds that modulate the activity of Wnt-5A or hFz5 and, thereby, modulate cardiomyocyte proliferation. The present invention provides methods for identifying compounds that alter one of more of the activities of the Wnt-5A signal transduction pathways, including but not limited to, modulation of cell proliferation. Specifically, compounds may be identified that promote cell proliferation. Alternatively, compounds that inhibit the Wnt-5A signal transduction pathways will be inhibitory for cell proliferation.

The present invention provides for methods for identifying a compound that activates the Wnt-5A signal transduction pathways comprising (i) contacting a cell expressing the hFz5 receptor with a test compound and measuring the level of hFz5 activity; (ii) in a separate experiment, contacting a cell expressing hFz5 protein with a vehicle control and measuring the level of hFz5 activity where the conditions are essentially the same as in part (i), and then (iii) comparing the level of hFz5 activity measured in part (i) with the level of hFz5 activity in part (ii), wherein an increased level of hFz5 activity in the presence of the test compound indicates that the test compound is a hFz5 activator.

The present invention also provides for methods for identifying a compound that inhibits the Wnt-5A signal transduction pathways comprising (i) contacting a cell expressing the hFz5 receptor with a test compound and the Wnt-5A protein and measuring the level of hFz5 activity; (ii) in a separate experiment, contacting a cell expressing the hFz5 receptor with Wnt-5A protein, where the conditions are essentially the same as in part (i) and then (iii) comparing the level of hFz5 activity measured in part (i) with the level of hFz5 activity in part (ii), wherein a decrease level of hFz5 activity in the presence of the test compound indicates that the test compound is a hFz5 inhibitor.

The ability of a test molecule to modulate the activity of the Wnt-5A signal transduction pathways maybe measured using standard biochemical and physiological techniques. For example, the effect on differentiation, survival, proliferation, or function of the cardiomyocytes may then be assessed.

In an embodiment of the invention, responses normally associated with activation of cell proliferation may be utilized. As described above, methods of measuring cell proliferation are well known in the art and most commonly include determining DNA synthesis characteristic of cell replication. Additionally, cells may be assayed to determine whether there are changes in levels, or modification, of proteins known to be associated with cell proliferation. Such proteins include, for example, cyclin D1, CDK4 or p107. In practice, for high throughput screens, microtitre plates conveniently can be utilized for quick and efficient screening of large quantities of test molecules.

Such screening assays may also involve the measurement of calcium transients. In one embodiment calcium imaging is used to measure calcium transients. For example, ratiometric dyes, such as fura-2, fluo-3, or fluo-4 are used to measure intracellular calcium concentration. The relative calcium levels in a population of cells treated with a ratiometric dye can be visualized using a fluorescent microscope or a confocal microscope. In other embodiments, the membrane potential across the cell membrane is monitored to assess calcium transients. For example, a voltage clamp may be used. In this method, an intracellular microelectrode is inserted into the cardiomyocyte. In one embodiment, calcium transients can be seen before observable contractions of the cardiomyocytes. In other embodiments calcium transients are seen either during, or after, observable contractions of cardiomyocytes. In another embodiment the cells are cultured in the presence of conditions wherein the cells do not beat, such as in the presence of a calcium chelator (e.g. EDTA or EGTA) and the calcium transients are measured.

In accordance with the invention, a cell based assay system can be used to screen for compounds that modulate the expression of Wnt-5A or hFz5 within a cell. Assays may be designed to screen for compounds that regulate Wnt-5A or hFz5 expression at either the transcriptional or translational level. In one embodiment, DNA encoding a reporter molecule can be linked to a regulatory element of the Wnt-5A or hFz5 gene and used in appropriate intact cells, cell extracts or lysates to identify compounds that modulate Wnt-5A or hFz5 gene expression. Such reporter genes may include but are not limited to chloramphenicol acetyltransferase (CAT), luciferase, β-glucuronidase (GUS), growth hormone, or placental alkaline phosphatase (SEAP). Such constructs are introduced into cells thereby providing a recombinant cell useful for screening assays designed to identify modulators of Wnt-5A or hFz5 gene expression.

Following exposure of the cells to the test compound; the level of reporter gene expression may be quantitated to determine the test compound's ability to regulate Wnt-5A or frizzeled expression. Alkaline phosphatase-assays are particularly useful in the practice of the invention as the enzyme is secreted from the cell. Therefore, tissue culture supernatant may be assayed for secreted alkaline phosphatase. In addition, alkaline phosphatase activity may be measured by calorimetric, bioluminescent or chemiluminescent assays such as those described in Bronstein, I. et al. (1994, Biotechniques 17: 172-177). Such assays provide a simple, sensitive easily automatable detection system for pharmaceutical screening.

[This section was added given that Wnt-5A inhibitors may be useful] In an embodiment of the invention, the level of Wnt-5A or fizzled expression can be modulated using antisense, ribozyme, or RNAi approaches to inhibit or prevent translation of Wnt-5A or hFz5 mRNA transcripts or triple helix approaches to inhibit transcription of the genes. Antisense and RNAi approaches involve the design of oligonucleotides (either DNA or RNA) that are complementary to Wnt-5A or hFz5 mRNA. The antisense or siNA oligonucleotides will be targeted to the complementary mRNA transcripts and prevent translation. Absolute complementarity, although preferred, is not required. One skilled in the art can ascertain a tolerable degree of mismatch by use of standard procedures to determine the melting point of the hybridized complex.

In a preferred embodiment of the invention, double-stranded short interfering nucleic acid (siNA) molecules may be designed to inhibit Wnt-5A expression. In one embodiment, the invention features a double-stranded siNA molecule that down-regulates expression of the Wnt-5A gene, wherein said siNA molecule comprises about 15 to about 28 base pairs.

In one embodiment, the invention features a double stranded short interfering nucleic acid (siNA) molecule that directs cleavage of a Wnt-5A RNA via RNA interference (RNAi), wherein the double stranded siNA molecule comprises a first and a second strand, each strand of the siNA molecule is about 18 to about 28 nucleotides in length, the first strand of the siNA molecule comprises nucleotide sequence having sufficient complementarity to the Wnt-5A RNA for the siNA molecule to direct cleavage of the Wnt-5-1 RNA via RNA interference, and the second strand of said siNA molecule comprises nucleotide sequence that is complementary to the first strand.

In one embodiment, the invention features a double stranded short interfering nucleic acid (siNA) molecule that directs cleavage of a Wnt-5A RNA via RNA interference (RNAi), wherein the double stranded siNA molecule comprises a first and a second strand, each strand of the siNA molecule is about 18 to about 23 nucleotides in length, the first strand of the siNA molecule comprises nucleotide sequence having sufficient complementarity to the Wnt-5A RNA for the siNA molecule to direct cleavage of the Wnt-5A RNA via RNA interference, and the second strand of said siNA molecule comprises nucleotide sequence that is complementary to the first strand.

In yet another embodiment of the invention, ribozyme molecules designed to catalytically cleave Wnt-5A or hFz5 mRNA transcripts can also be used to prevent translation of Wnt-5A or hFz5 mRNA and expression of Wnt-5A or hFz5. (See, e.g., PCT International Publication WO90/11364, published Oct. 4, 1990; Sarver et al., 1990, Science 247:1222-1225). Alternatively, endogenous Wnt-5A or hFz5 gene expression can be reduced by targeting deoxyribonucleotide sequences complementary to the regulatory region of the Wnt-5A or hFz5 genes (i.e., the Wnt-5A or hFz5 promoter and or enhancers) to form triple helical structures that prevent transcription of the Wnt-5A or hFz5 gene in targeted hematopoietically-derived cells in the body. (See generally, Helene, C. et al., 1991, Anticancer Drug Des. 6:569-584 and Maher, L J, 1992, Bioassays 14:807-815).

The oligonucleotides of the invention, i.e., antisense, ribozyme and triple helix forming oligonucleotides, may be synthesized by standard methods known in the art, e.g., by use of an automated DNA synthesizer (such as are commercially available from Biosearch, Applied Biosystems, etc.). Alternatively, recombinant expression vectors may be constructed to direct the expression of the oligonucleotides of the invention. Such vectors can be constructed by recombinant DNA technology methods standard in the art. In a specific embodiment, vectors such as viral vectors may be designed for gene therapy applications where the goal is in vivo expression of inhibitory oligonucleotides in targeted cells.

The assays described above can identify compounds which modulate Wnt-5A signal transduction activity. For example, compounds that affect Wnt-5A signal transduction activity include but are not limited to compounds that bind to Wnt-5A or hFz5, and either activate the signal transduction activities or block the signal transduction activities. Alternatively, compounds may be identified that do not bind directly to Wnt-5A or hFz5 but are capable of altering signal transduction activity by altering the activity of a protein that regulates Wnt-5A signal transduction activity.

The compounds which may be screened in accordance with the invention include, but are not limited to, small organic or inorganic compounds, peptides, antibodies and fragments thereof, and other organic compounds e.g., peptidomimetics) that bind to hFz5 and either mimic the activity triggered by Wnt-5A (i.e., agonists) or inhibit the activity triggered by Wnt-5A (i.e., antagonists). Compounds that enhance Wnt-5A signal transduction activities, i.e., agonists, or compounds that inhibit Wnt-5A signal transduction activities, i.e., antagonists, in the presence or absence of Wnt-5A will be identified. Compounds that bind to proteins and alter/modulate the Wnt-5A signal transduction activities will be identified.

Compounds may include, but are not limited to, peptides such as, for example, soluble peptides, including but not limited to members of random peptide libraries (see, e.g., Lam, K. S. et al., 1991, Nature 354:82-84; Houghten, R. et al., 1991, Nature 354:84-86); and combinatorial chemistry-derived molecular library made of D- and/or L-configuration amino acids, phosphopeptides (including, but not limited to, members of random or partially degenerate, directed phosphopeptide libraries; (see, e.g., Songyang, Z. et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope binding fragments thereof), and small organic or inorganic molecules.

Other compounds which maybe screened in accordance with the invention include but are not limited to small organic molecules that affect the biological activity, or expression, of the Wnt-5A or frizzled genes or some other genes involved in the Wnt-5A signal transduction pathway (e.g., by interacting with the regulatory region or transcription factors involved in gene expression); or such compounds that affect the activities of Wnt-5A or frizzled, or the activity of some other factor involved in modulating Wnt-5A/frizzled signal transduction activities.

5.2. Use of Scaffolds for Promotion of Cardiomyocyte Proliferation

According to another aspect of the invention, a method for regenerating myocardium in a mammal is provided, comprising attracting native stem cells to the myocardium wherein said native stem cells induce native cardiomyocytes to enter the cell cycle. The native stem cells may be attracted to the myocardium by administration of a scaffold to the region of the heart in need of repair. In another embodiment of the invention, a portion of the myocardium is excised and replaced with a scaffold.

As demonstrated herein a variety of different scaffolds may be used successfully in the practice of the invention. Such scaffolds are typically administered to the subject in need of treatment as a transplanted patch. Preferred scaffolds include, but are not limited to biological, degradable scaffolds. In an embodiment of the invention, the scaffold is derived from porcine urinary bladder. Alternatively, in a preferred embodiment of the invention the scaffold is derived from bovine pericardium. In a specific embodiment of the invention, Veritas® which is derived from bovine pericardium may be utilized. Additionally, such scaffolds may be supplemented with additional components capable of stimulating cardiomyocyte proliferation. Such components, include but are not limited to, stem cells, components of stem cell conditioned media, modulators of the Wnt-5A signal transduction pathway and/or Wnt-5A protein.

Stem cells can also be incorporated or embedded within scaffolds which are recipient-compatible and which degrade into products which are not harmful to the recipient. These scaffolds provide support and protection for stem cells that are to be transplanted into the recipient subjects. Natural and/or synthetic biodegradable scaffolds are examples of such scaffolds. Accordingly, the present invention provides methods for promoting cardiac repair, wherein stem cells are incorporated within scaffolds, prior to transplantation into a subject in need of cardiac repair. In a preferred embodiment of the invention, the stem cells are mesenchymal stem cells.

Alternatively, the scaffold may be engineered to contain agents capable of attracting native stem cells to said scaffold. For example, the scaffold may be engineered to contain c-kit antibodies capable of binding to native stem cells. The scaffold may also be engineered to contain granulocyte colony-stimulating factor or stem cell factor to attract native stem cells.

In yet another embodiment of the invention, the stem cells may be genetically engineered to express biological agents capable of stimulating cardiomyocyte proliferation prior to incorporation into the scaffold. For example, the stem cells may be genetically engineered to express a modulator of Wnt-5A signal transduction pathways. Such modulators include, for example, activators of the signal transduction pathway, such as for example, Wnt-5A.

In yet another embodiment of the invention, scaffolds may be placed in contact with stem cell conditioned media prior to transplantation of the scaffold into the subject in need of cardiac repair. As demonstrated herein, stem cell conditioned media contains biologically active components capable of stimulating cardiomyocyte proliferation. By placing the conditioned media in contact with the scaffold, the biologically active components of the media should become incorporated into the scaffold.

The present invention further provides methods wherein a scaffold is formed which incorporates modulators of the Wnt-5A signal transduction pathways. As demonstrated herein, Wnt-5A is a molecule capable of stimulating cardiomyocyte proliferation. Accordingly, scaffolds can be formed containing the Wnt-5A protein incorporated therein. Such scaffolds may be used to treat a subject in need of cardiac repair.

Natural biodegradable scaffolds include collagen, fibronectin, and laminin scaffolds. Suitable synthetic material for a cell transplantation scaffold must be biocompatible to preclude migration and immunological complications, and should be able to support extensive cell growth and differentiated cell function. It must also be resorbable, allowing for a completely natural tissue replacement. The scaffold should be configurable into a variety of shapes and should have sufficient strength to prevent collapse upon implantation. Recent studies indicate that the biodegradable polyester polymers made of polyglycolic acid fulfill all of these criteria, as described by Vacanti, et al. J. Ped. Surg. 23:3-9 (1988); Cima, et al. Biotechnol. Bioeng. 38:145 (1991); Vacanti, et al. Plast. Reconstr. Surg. 88:753-9 (1991). Other synthetic biodegradable support scaffolds include synthetic polymers such as polyanhydrides, polyorthoesters, and polylactic acid.

Support scaffolds into which the stem cells can be incorporated or embedded include scaffolds which are recipient-compatible and which degrade into products which are not harmful to the recipient. These scaffolds provide support and protection for stem cells and differentiated cells in vivo and are, therefore, the preferred form in which such cells are transplanted into the recipient subjects.

Attachment of the cells to the polymer may be enhanced by coating the polymers with compounds such as basement membrane components, agar, agarose, gelatin, gum arabic, collagens types I, II, III, IV and V, fibronectin, laminin, glycosaminoglycans, mixtures thereof, and other materials known to those skilled in the art of cell culture. All polymers for use in the scaffold must meet the mechanical and biochemical parameters necessary to provide adequate support for the cells with subsequent growth and proliferation. The polymers can be characterized with respect to mechanical properties such as tensile strength using an Instron tester, for polymer molecular weight by gel permeation chromatography (GPC), glass transition temperature by differential scanning calorimetry (DSC) and bond structure by infrared (IR) spectroscopy, with respect to toxicology by initial screening tests involving Ames assays and in vitro teratogenicity assays, and implantation studies in animals for immunogenicity, inflammation, release and degradation studies.

One of the advantages of a biodegradable polymeric scaffold is that angiogenic and other bioactive compounds can be incorporated directly into the support scaffold so that they are slowly released as the support scaffold degrades in vivo. Factors, including nutrients, growth factors, inducers of proliferation or de-differentiation (i.e., causing differentiated cells to lose characteristics of differentiation and acquire characteristics such as proliferation and more general function), products of secretion, immunomodulators, inhibitors of inflammation, regression factors, biologically active compounds which enhance or allow ingrowth of nerve fibers, hyaluronic acid, and drugs, which are known to those skilled in the art and commercially available with instructions as to what constitutes an effective amount, from suppliers such as Collaborative Research, Sigma Chemical Co., Vascular growth factors such as vascular endothelial growth factor (VEGF), epidermal growth factor (EGF), and heparin binding epidermal growth factor like growth factor (HB-EGF), could be incorporated into the scaffold or provided in conjunction with the scaffold. Similarly, polymers containing peptides such as the attachment peptide RGD (Arg-Gly-Asp) can be synthesized for use in forming scaffolds (see e.g U.S. Pat. Nos. 4,988,621, 4,792,525, 5,965,997, 4,879,237 and 4,789,734).

In another example, the cells may be transplanted in a gel scaffold (such as Gelfoam from Upjohn Company) which polymerizes to form a substrate in which the stem cells can grow. A variety of encapsulation technologies have been developed (e.g. Lacy et al., Science 254:1782-84 (1991); Sullivan et al., Science 252:718-712 (1991); WO 91/10470; WO 91/10425; U.S. Pat. No. 5,837,234; U.S. Pat. No. 5,011,472; U.S. Pat. No. 4,892,538). During open surgical procedures, involving direct physical access to the damaged tissue and/or organ, all of the described forms of stem cell delivery preparations are available options. These cells can be repeatedly transplanted at intervals until a desired therapeutic effect is achieved.

5.4. Uses and Administration of the Compositions of the Invention

The present invention provides methods and compositions which may be used therapeutically for treatment of various diseases associated with cardiac disorders. The term “cardiac disorder” as used herein refers to diseases that result from any impairment in the heart's pumping function. This includes, for example, impairments in contractility, impairments in ability to relax (sometimes referred to as diastolic dysfunction), abnormal or improper functioning of the heart's valves, diseases of the heart muscle (sometimes referred to as cardiomyopathy), diseases such as angina and myocardial ischemia and infarction characterized by inadequate blood supply to the heart muscle, infiltrative diseases such as amyloidosis and hemochromatosis, global or regional hypertrophy (such as may occur in some kinds of cardiomyopathy or systemic hypertension), and abnormal communications between chambers of the heart (for example, atrial septal defect). For further discussion, see Braunwald, Heart Disease: a Textbook of Cardiovascular Medicine, 5th edition, W B Saunders Company, Philadelphia Pa. (1997) (hereinafter Braunwald). The term “cardiomyopathy” refers to any disease or dysfunction of the myocardium (heart muscle) in which the heart is abnormally enlarged, thickened and/or stiffened. As a result, the heart muscle's ability to pump blood is usually weakened. The disease or disorder can be, for example, inflammatory, metabolic, toxic, infiltrative, fibroplastic, hematological, genetic, or unknown in origin. There are two general types of cardiomyopathies: ischemic (resulting from a lack of oxygen) and nonischemic. Other diseases include congenital heart disease which is a heart-related problem that is present since birth and often as the heart is forming even before birth or diseases that result from myocardial injury which involves damage to the muscle or the myocardium in the wall of the heart as a result of disease or trauma. Myocardial injury can be attributed to many things such as, but not limited to, cardiomyopathy, myocardial infarction, or congenital heart disease. Specific cardiac disorders to be treated also include congestive heart failure, ventricular or atrial septal defect, congenital heart defect or ventricular aneurysm. The cardiac disorder may be pediatric in origin. The cardiac disorder may require ventricular reconstruction.

The present invention provides for methods of stimulating cardiomyocyte proliferation comprising contacting cardiomyocytes with an effective amount of stem cells or conditioned media derived from stem cells. Accordingly, the present invention provides a method for treating a subject afflicted with a cardiac disorder comprising administering stem cells to said subject. The stem cells may be administered and/or transplanted to a subject suffering from a cardiac disease in any fashion know to those of skill in the art. Stem cells to be administered include, but are not limited to, human mesenchymal stem cells. Additionally, the stem cells to be transplanted may be genetically engineered to express molecules capable of stimulating cardiomyocyte proliferation such as, for example, Wnt-5A.

According to another aspect of the invention, a method for treating a subject afflicted with a cardiac disorder, in vivo, is provided, comprising (i) producing a solution comprising media conditioned from the culturing, in vitro, of stem cells and (ii) administering the solution of step (i) to the subject, thereby treating the cardiac disorder in the subject. In another embodiment of the invention step (i) may be performed in the presence of cardiomyocytes. In a preferred embodiment of the invention, the stem cells are mesenchymal stem cells.

In an embodiment of the invention, components of the conditioned media may be administered via a scaffold. Alternatively, the conditioned media may be administered via an injection into the blood stream, coronary artery, coronary vein, myocardium. Or pericardial space.

Additionally, the present invention provides methods for stimulation of cardiomyocyte proliferation comprising administration to the area of the myocardium in need of repair a scaffold in an amount sufficient to stimulate cardiomyocyte proliferation. Such scaffolds have been demonstrated to function through attraction of endogenous stem cells to the region of the myocardium in need of repair. Additionally, scaffolds that have been supplemented with components known to stimulate the proliferation of cardiomyocytes may be administered to the myocardium.

In yet another embodiment of the invention, modulators of the Wnt-5/hFz5 signal transduction pathway may be used to treat subjects suffering from a cardiac disorder. In a preferred embodiment, the Wnt-5A protein is administered to the subject in need of treatment.

Various delivery systems are known and can be used to administer a compound capable of regulating cardiomyocyte proliferation. Such compositions may be formulated in any conventional manner using one or more physiologically acceptable carriers optionally comprising excipients and auxiliaries. Proper formulation is dependent upon the route of administration chosen.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. The composition can be formulated as a suppository, with traditional binders and carriers such as triglycerides. Oral formulation can include standard carvers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical sciences” by E.W. Martin. Such compositions will contain a therapeutically effective amount of the therapeutic compound, preferably in purified form, together with a suitable amount of carrier so as to provide the form for proper administration to the patient. The formulation should suit the mode of administration.

The compositions of the invention can be administered by injection into a target site of a subject, preferably via a delivery device, such as a tube, e.g., catheter. In a preferred embodiment, the tube additionally contains a needle, e.g., a syringe, through which the compositions can be introduced into the subject at a desired location.

The compositions may be inserted into a delivery device, e.g., a syringe, in different forms. For example, the compositions of the invention can be suspended in a solution contained in such a delivery device. As used herein, the term “solution” includes a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art.

The compositions of the invention may be administered systemically (for example intravenously) or locally (for example directly into a myocardial defect under echocardiogram guidance, or by direct application under visualization during surgery). For such injections, the compositions may be in an injectible liquid suspension preparation or in a biocompatible medium which is injectible in liquid form and becomes semi-solid at the site of damaged tissue. A conventional intra-cardiac syringe or a controllable endoscopic delivery device can be used so long as the needle lumen or bore is of sufficient diameter (e.g. 30 gauge or larger) that shear forces will not damage the cells being delivered.

In a specific embodiment, it may be desirable to administer the compositions of the invention locally to a specific area of the body; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non porous, or gelatinous material, including membranes, such as silastic membranes, or fibers.

The appropriate concentration of the composition of the invention which will be effective in the treatment of a particular cardiac disorder or condition will depend on the nature of the disorder or condition, and can be determined by one of skill in the art using standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses maybe extrapolated from dose response curves derived from in vitro or animal model test systems. Additionally, the administration of the compound could be combined with other known efficacious drugs if the in vitro and in vivo studies indicate a synergistic or additive therapeutic effect when administered in combination.

The progress of the recipient receiving the treatment may be determined using assays that are designed to test cardiac function. Such assays include, but are not limited to ejection fraction and diastolic volume (e.g., echocardiography), PET scan, CT scan, angiography, 6-minute walk test, exercise tolerance and NYHA classification.

### 6. EXAMPLE

### STEM CELL MEDIATED STIMULATION OF CARDIOMYOCYTE PROLIFERATION

The subsection below describes data demonstrating that implantation of extracellular scaffolds are capable of stimulating proliferation of cardiomyocytes.

6.1. Materials and Methods

Human mesenchymal stem cells were obtained from BioWhittaker/Cambrex Inc. Cyclin-D1, Ki-67 and c-kit antibodies were purchased from Santa Cruz Biotechnology Inc. Antibody for α-sarcomeric actinin was purchased from Sigma. Urinary bladder extracellular scaffold membrane (ECM) was generously provided by Dr. Stephan Badylak (University of Pittsburgh).

To introduce an amputation wound in the canine heart a full thickness portion of myocardium of right ventricle approximately 15×10 mm was excised and replaced with membrane made of ECM or Dacron in adult mongrel dogs. All animal received humane care in accordance with the “Principles of Laboratory Animal Care” formulated by the National Society for Medical Research and the “Guide for the Care and Use of Laboratory Animals” of National Academy of Sciences (NIH publication No. 85-23) and treated according to protocol IACUC#20031326 approved by the Animal Care and Use Committee at SUNY Stony Brook,

For in vitro experiments canine ventricular cardiomyocytes were isolated in Tyrode's solution as described (18) supplied with 10 nM insulin and placed on poly-D-lysine—laminin coated 35 mm cell culture dishes or in Lab-Tek II CC2 chamber slides (ED Biosciencies). Myocytes were maintained in a humidified atmosphere of 5% CO2 at 37° C. After 3-4 hours, thyroid solution was replaced with serum free DMEM media containing 10 nM insulin. After 9-12 hours, cardiomyocytes cells were washed twice with DMEM and supplied with hMSCs in DMEM containing 5% fetal bovine serum to produce 50% confluent monolayer of hMSCs. Media was changed once every four days.

6.2. Results

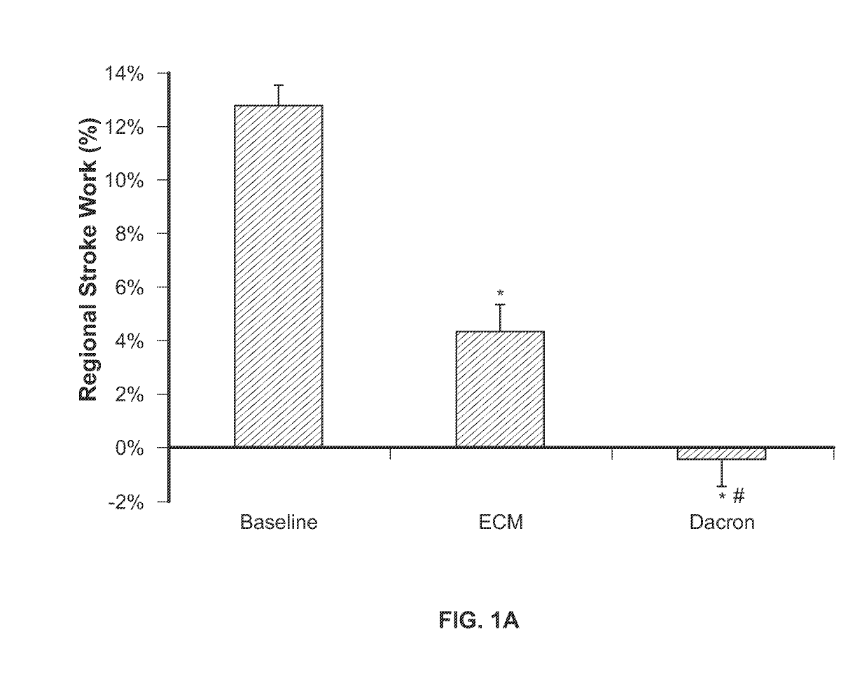

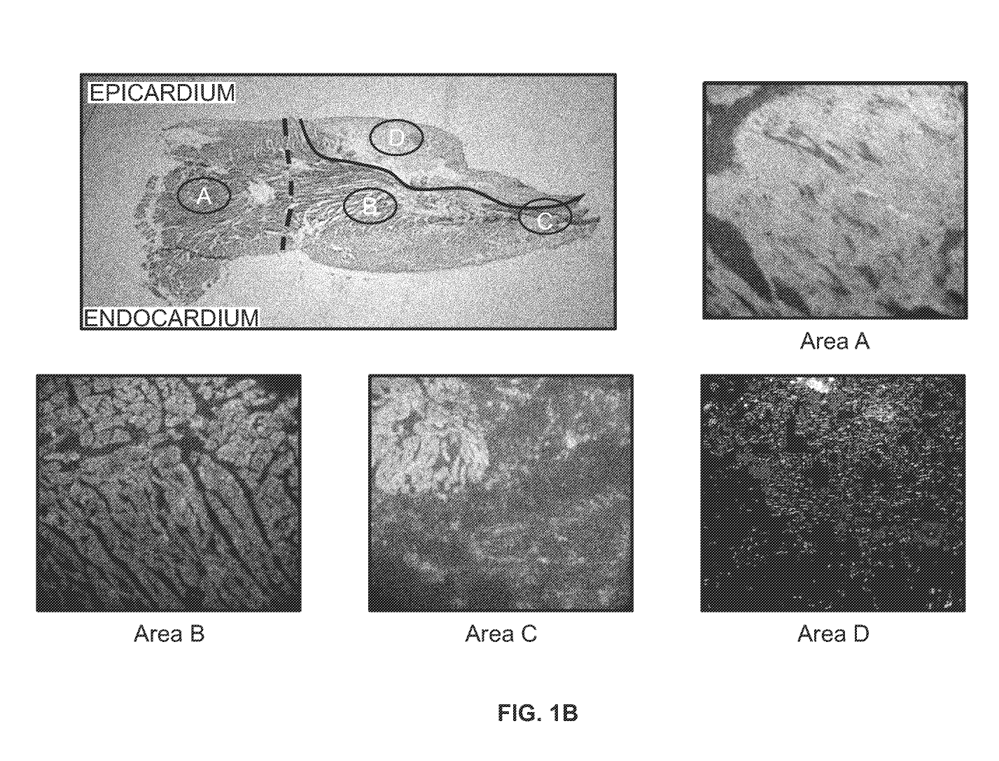

To investigate how generally healthy mammalian heart will respond to similar injuries a small (less than 5% of area) full thickness region of the canine right ventricle was excised and replaced with a patch made of either extracellular scaffold (ECM) prepared from swine bladder (Badylak, S. F., 2002, Cell Dev. Biol. 13, 377-383 (2002) or Dacron synthetic material. Regional heart performance was assayed eight weeks after implantation as previously described (Gaudette, G. R. et al., 2002, Cardiovascular Engineering: An International Journal 2, 129-137; Gaudette, G. R. et al., . . . , 2001, Ann. Biomed. Eng 29, 775-780 (2001). Non-implant regions of the heart (n=4) displayed a regional stroke work of 13±1% (normalized to developed pressure and end diastolic area), whereas Dacron implants (n=4) had 0±1% regional stroke work. Regional function was significantly better in the ECM implanted hearts (n=4) with a regional stroke work of 4±1% (FIG. 1A), suggesting that contractile function in the implant region was partially restored. Microscopic examination revealed that the myocardium was partially regenerated in the ECM implant (FIG. 1B). Cells with myocyte morphology that stained positively for α-sarcomeric actinin (α-SA) were located at the endocardial side of the ECM implant. In addition, a gradient of thickness from the periphery to the center of the patch was observed. No myocytes were found in Dacron implants. The absence of myocardial regeneration with the Dacron implants suggested that the microenvironment resulting from the scaffold implantation may play a crucial role in the process of regeneration

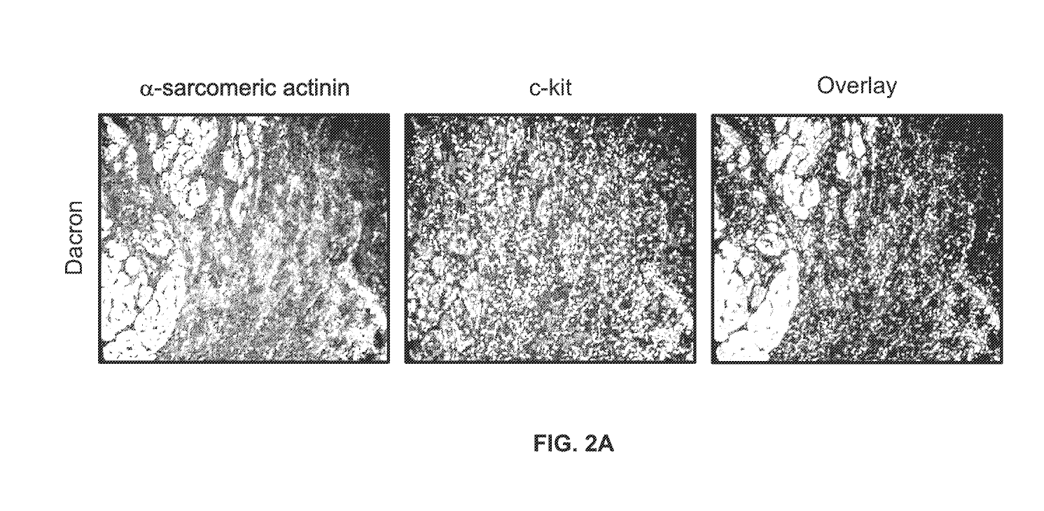

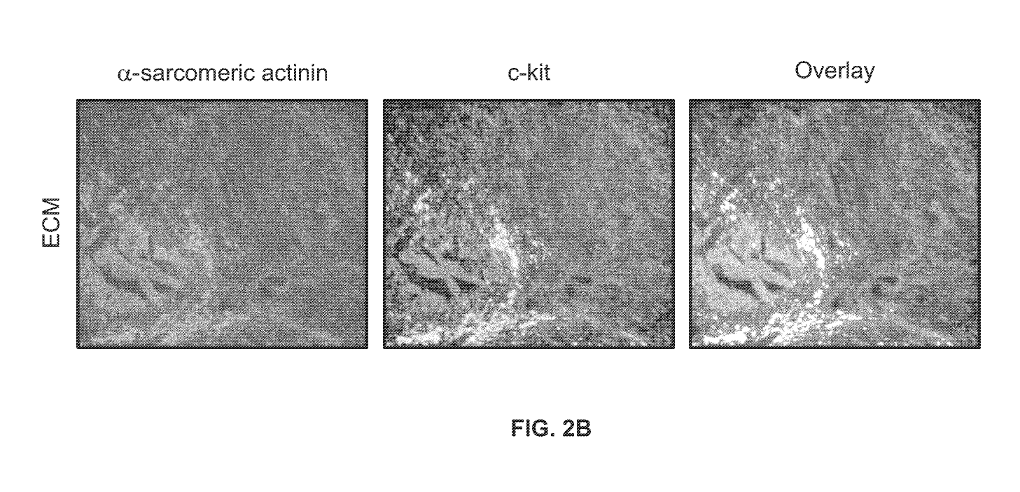

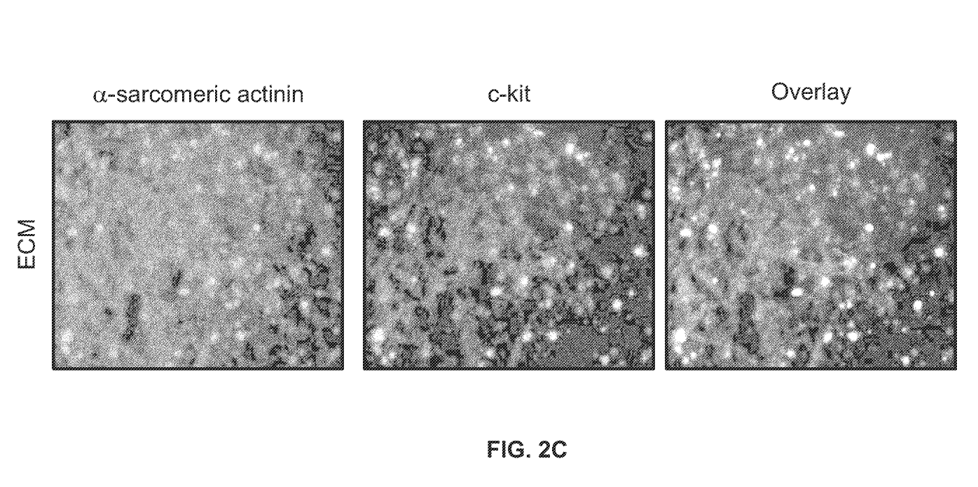

ECM assisted regeneration of canine myocardium demonstrates that like amphibians or zebrafish the mammalian heart can regenerate amputated myocardium. However, this process requires the presence of “healthy” extracellular scaffold. To investigate the possible mechanisms of regeneration animals were assayed at two weeks post implantation of either Dacron oz ECM for the presence of cells that are c-kit positive, as Lin-c-kit+ stem cells have been repotted to play a pivotal role in myocardial regeneration (Beltrami, A. P. et al., 2003,Cell 114, 763-776; Orlic, D. et al., 2003, Pediatr. Transplant 7 Suppl. 3, 86-88). ECM implants were found to be populated with c-kit+ cells gravitating to the mid-myocardium and endocardial regions of the implant. Adjacent host myocardium did not contain c-kit+ cells, suggesting that the stem cells may be derived from the blood stream. Proliferation of c-kit+ cells occurred at the border area of the implant and host myocardium FIG. 2B). In contrast, the Dacron implants were depleted of c-kit+ cells (FIG. 2A), although a small number could be found after thorough examination. Furthermore, in the ECM implants, many c-kit+ stem cells also stained positive far alpha-sarcomeric actinin (α-SA), independent of whether they made contact with the host myocardium (FIG. 2C). This suggests that direct contact with cardiomyocytes is not required for α-SA expression. Regeneration of myocardium in the presence of ECM correlates with the presence of c-kit+ cells. However, recent studies have shown that c-kit+ stem cells adopt mature hematopoetic fates in myocardium and do not transdifferentiate in cardiomyocytes (Balsam, L. B. et al., 2004, Nature 428, 668-673; Murry, C. E. et al., 2004, Nature 428, 664-668). This suggest that differentiation of c-kit+cells into myocytes might not be involved in the repair observed in the ECM implants, suggesting that myocardial regeneration occurs though a different mechanism.

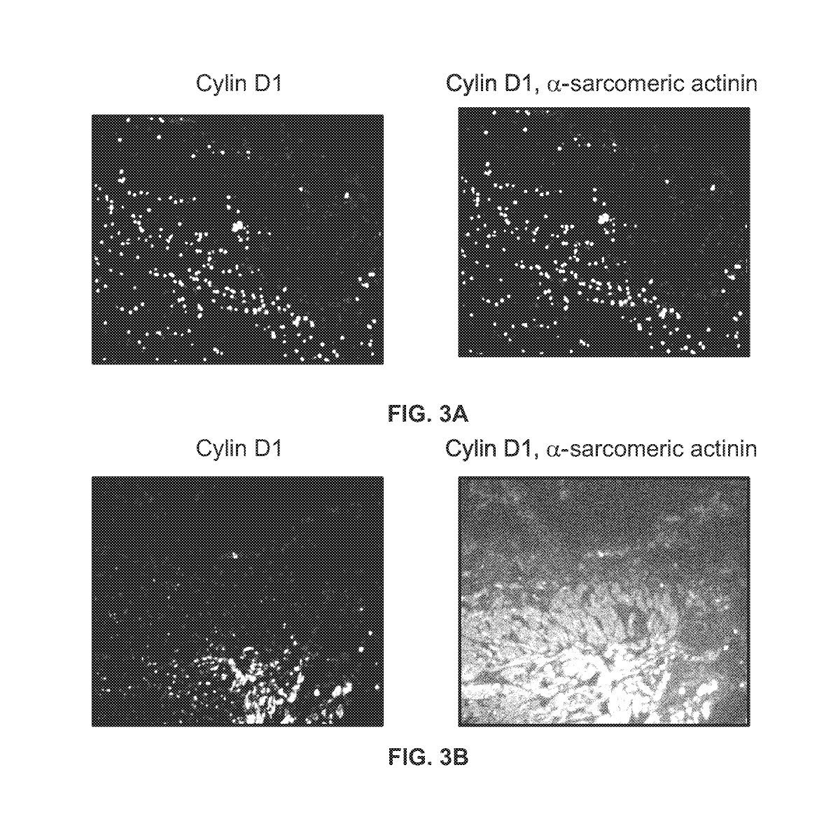

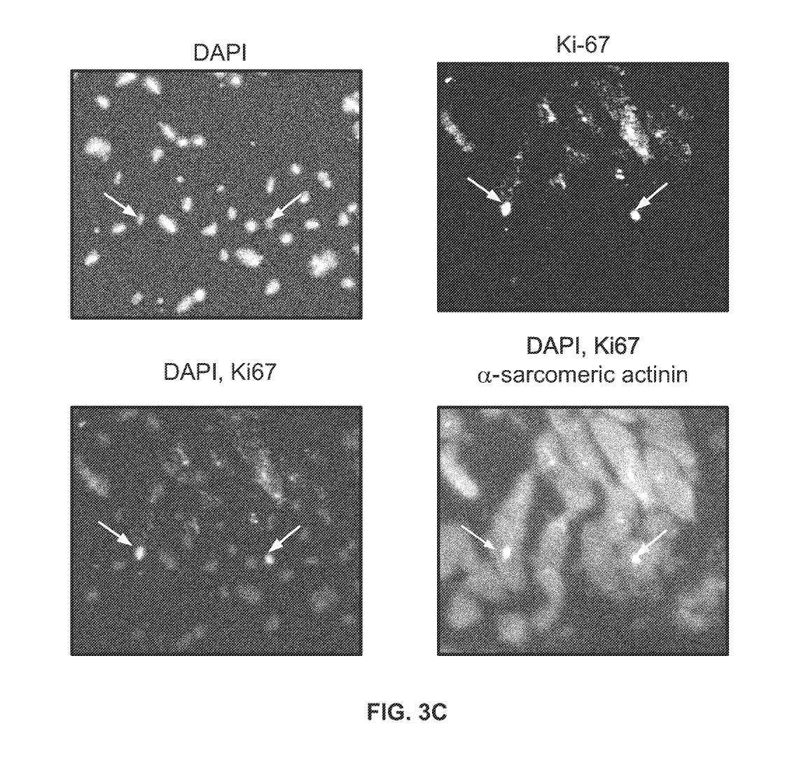

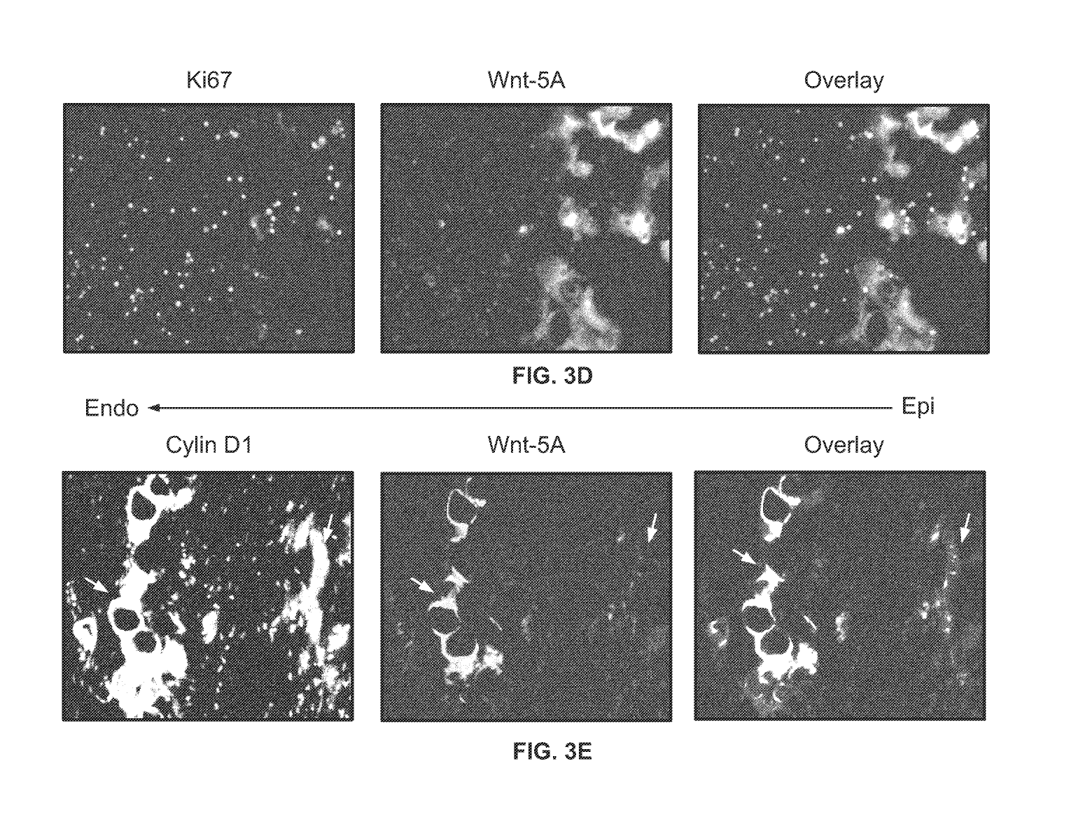

In considering alternatives, amphibian myocardium regenerates as a result of mitotic division of cardiomyocytes. To evaluate this mechanism of regeneration implants were examined for expression of two markers of cell division Ki-67 and cyclin D1. The two week ECM implants did not show improvement in regional contraction or myocardium reconstitution that is seen in the implant regions at eight weeks. Eight week ECM implants were essentially free of c-kit+ cells. Host myocardium adjacent to the implant at eight weeks area A in FIG. 1B) did not show staining for Ki-67 or cyclin D1, demonstrating that this region was composed of mitotically silent cardiomyocytes. A similar result was obtained for the internal area of regenerated myocardium (area B in FIG. 1B). A layer of cells at the epicardial surface of the implant area D in FIG. 1B) contained cyclin D1 positive cells (FIG. 3A). Cardiomyocytes at the tip of the regeneration cone (area C in FIG. 1B) were found to be cyclin D1 (FIG. 3B) and Ki-67 positive (FIG. 3C). These results show that mitotic expansion of myocytes might be part of the mechanism of myocardial regeneration in the ECM implants. This suggests that regeneration of the amputated mammalian heart might follow the mechanism of amphibian heart regeneration. The data obtained also correlate with heart regeneration in MRL mice (Leferovich, J. M. et al., 2001, Proc. Natl . Acad. Sci. U.S. A 98, 9830-9835). In the case of MRL mice, the mitotic index of myocytes (10-20%) during regeneration of cryogenically injured heart was close to that of amphibians(4). MRL mice regenerate wounds without forming scars, presumably due to an altered mechanism of ECM remodeling (Leferovich, J. M. et al., 2001, Proc. Natl. Acad. Sci. U.S. A 98, 9830-9835). ECM assisted myocardial regeneration shows that normal extracellular scaffold attracts stem cells and later gives raise to a population of mitotically competent cardiomyocytes.

Cardiomyocytes leave the cell cycle shortly after birth and lose markers of cell division. Expression of Ki-67 and cyclin D1, the markers of mitotically competent cells, suggests that cardiomyocytes were exposed to stimulators of mitotic proliferation. To study the signals which support cellular proliferation, the distribution of Wnt-5A+a stimulator of cyclin D1 expression (Shtutman, M. et al., 1999, Proc. Natl. Acad. Sci. U.S. A 96, 5522-5527; Tetsu, O. & McCormick, F., 1999, Nature 398, 442-426), was examined. Wnt-5A+ cells were located (FIG. 3D) above the layer of dividing cells (FIG. 3A) at the epicardial surface in the eight week ECM implant. A second layer of Wnt-5A+ cells was identified under the layer of proliferating cardiomyocytes (FIG. 3B) at the endocardial surface of the implant (FIG. 3E). Wnt-5A+ cells were not found in the myocardium of control dogs or in the area of host myocardium adjacent to the site of surgery. These data suggest that population of the ECM implant with stem cells establishes an environment resulting in the expression of signalling factors that are not present in the normal myocardium.

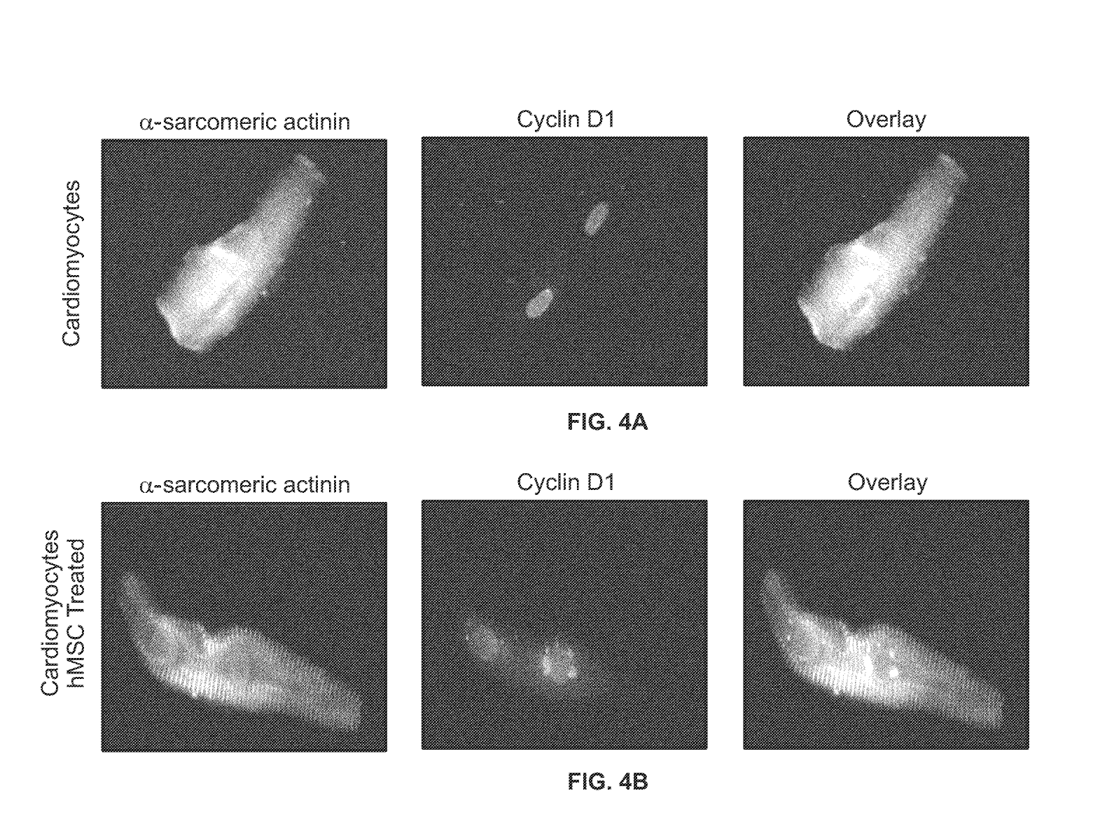

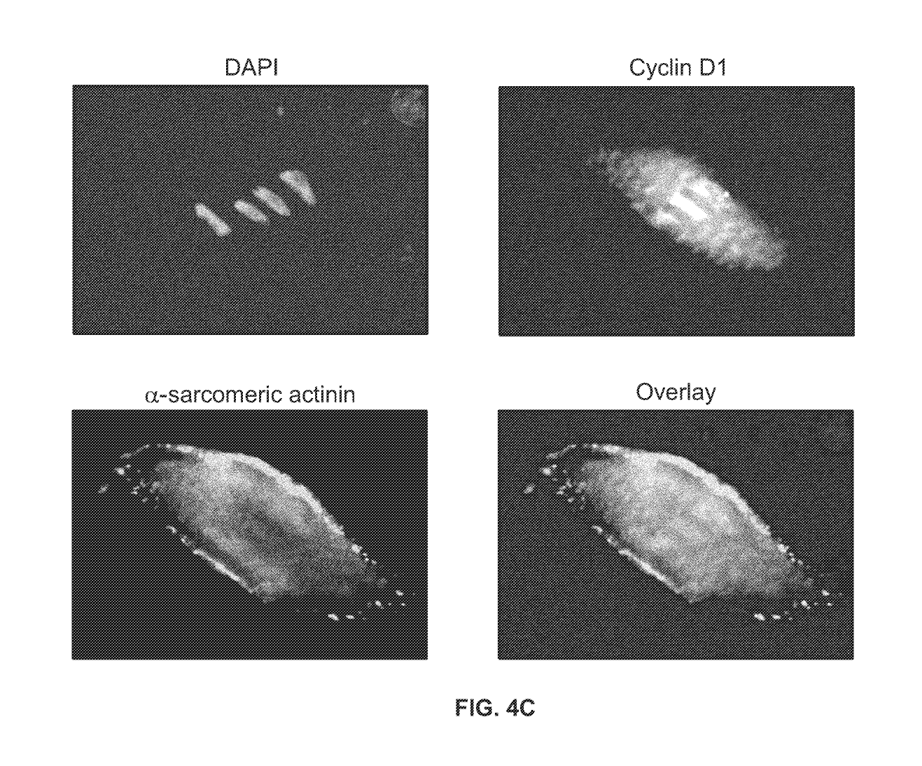

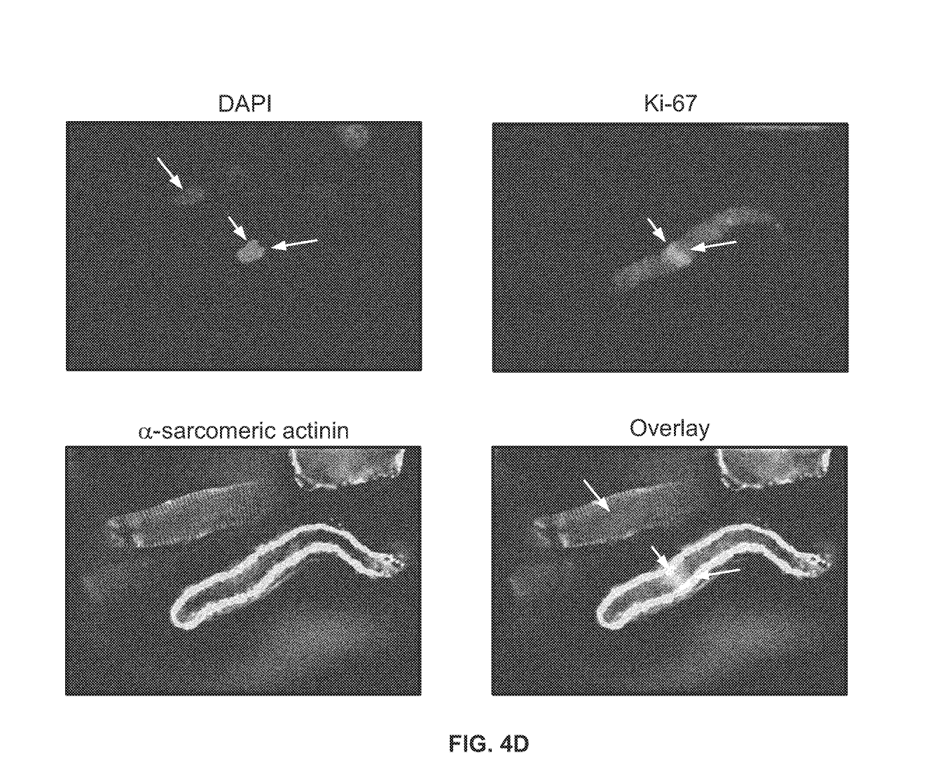

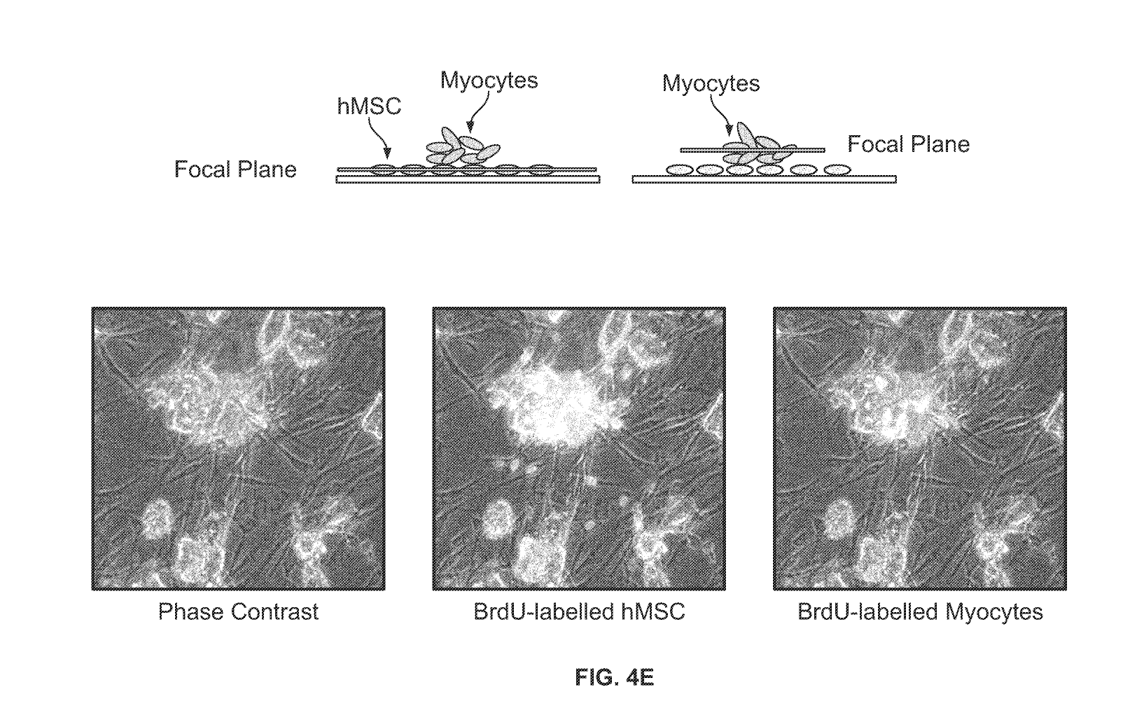

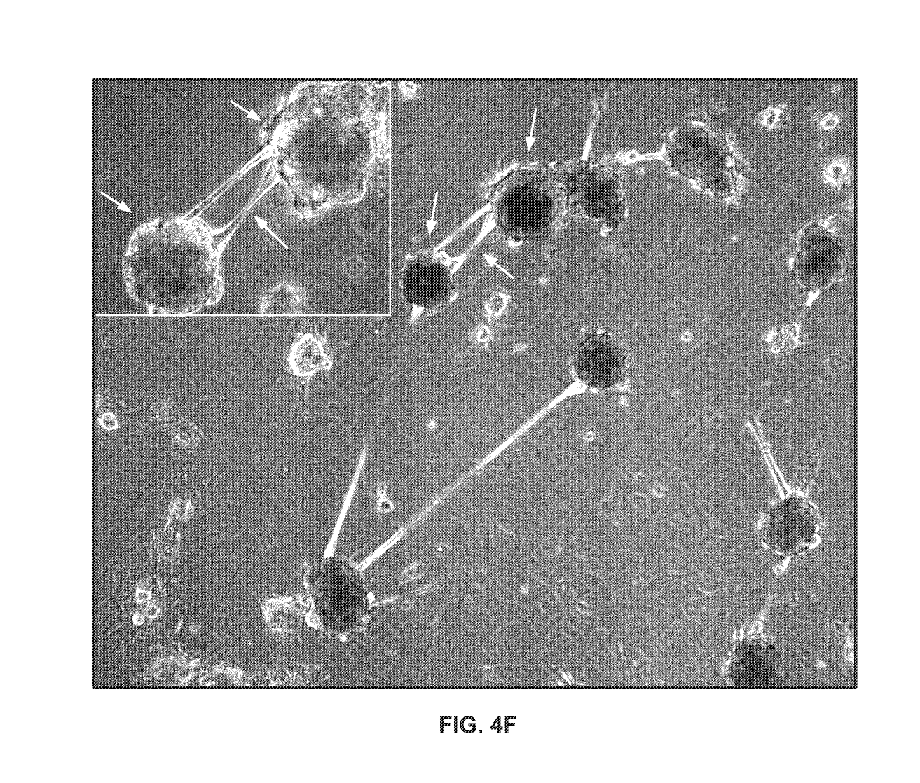

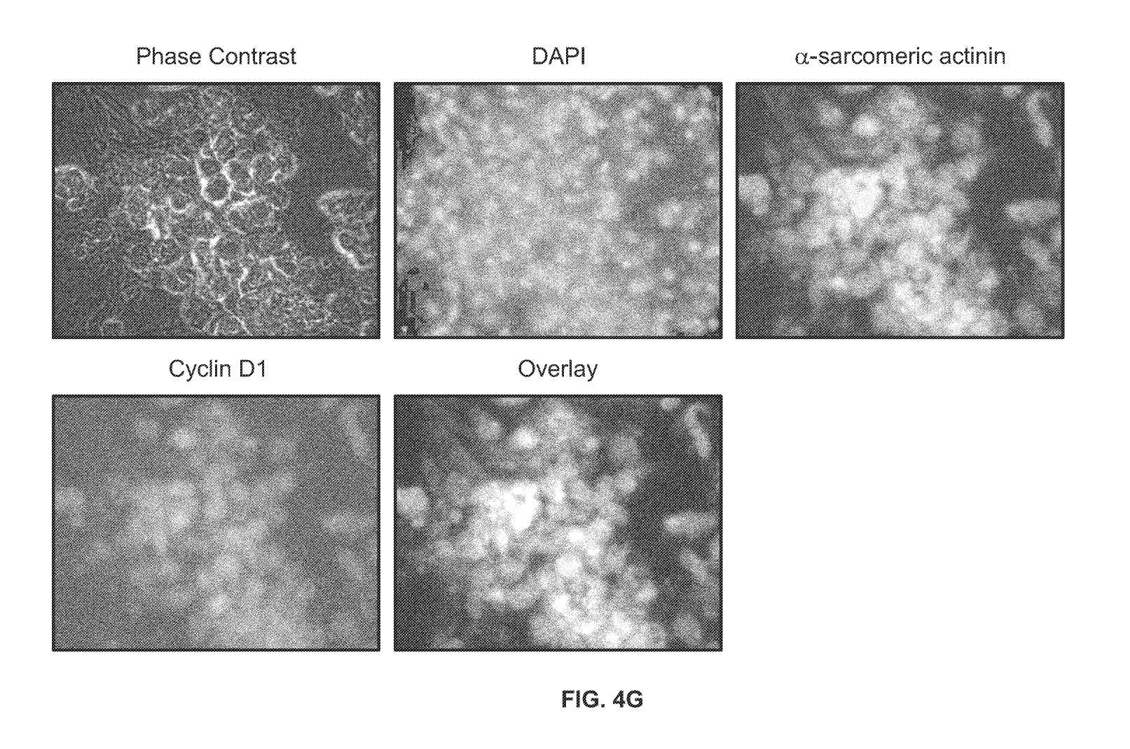

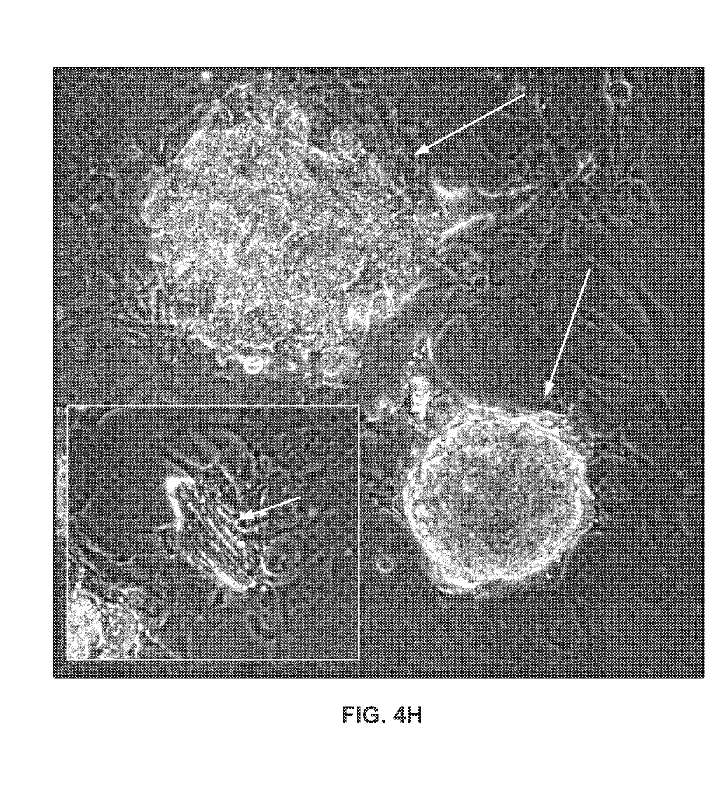

To mimic myocyte exposure to factors produced by stem cells in ECM implants, cardiomyocytes isolated from canine ventricle were co-cultured with human mesenchymal stem cells (hMSCs), without ECM. Mesenchymal stem cells have been shown to regenerate myocardium, although through a mechanism other than transdifferentiation into cardiomyocytes (Rodic, N., et al., 2004,Trends Mol. Med 10, 93-96). These hMSCs produce a variety of signalling factors, including a set of Wnt proteins (Doi, M. et al., 2002, Biochemical and Biophysical Research Communications 290, 381-390;Gregory, C.A., et al., 2003, J. Biol. Chem. 278, 28067-28078). After 3-4 days of co-culture, expression of cyclin D1 was detected in cardiomyocytes, whereas control cardiomyocytes, maintained in the absence of stems cells, remain cyclin D1 negative (FIG. 4A,B).

Intermediates of cell division were detected after 4-5 days of co-culture with hMSCs among the cyclin D1 and Ki-67 positive cardiomyocytes (FIG. 4C,D). After ten days co-cultured with hMSCs, cardiomyocytes formed colonies that included cells stained positive for DNA synthesis with BrdU (FIG. 4E). Two week old colonies of cardiomyocytes were often interconnected by spontaneously contracting myocytes (FIG. 4F). During the next thirty days colonies of myocytes proliferated and formed conical structures on the surface of the hMSCs that included dozens of cyclin D1 and Ki-67 positive cardiomyocytes (FIG. 4G). These colonies were viable for at least three months.

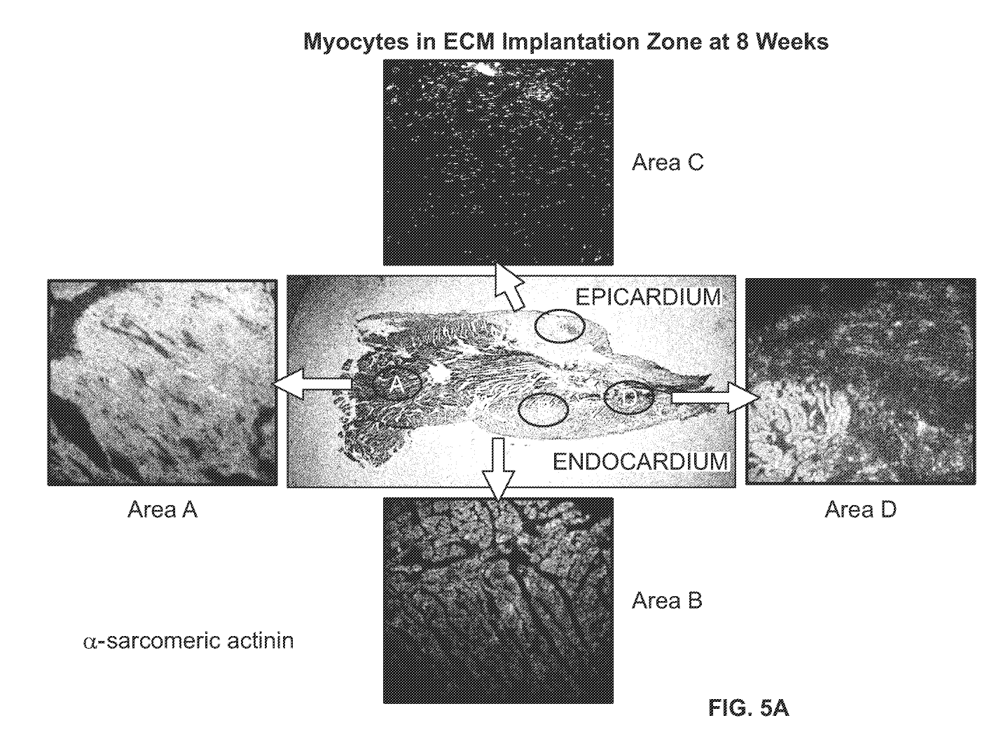

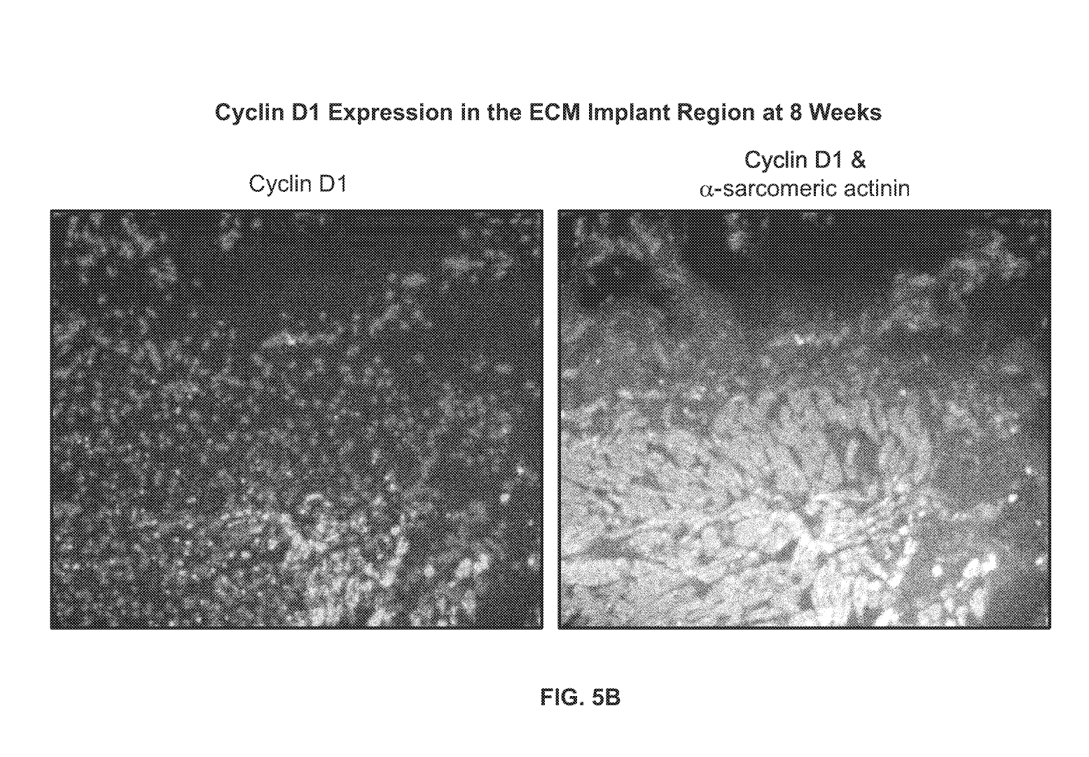

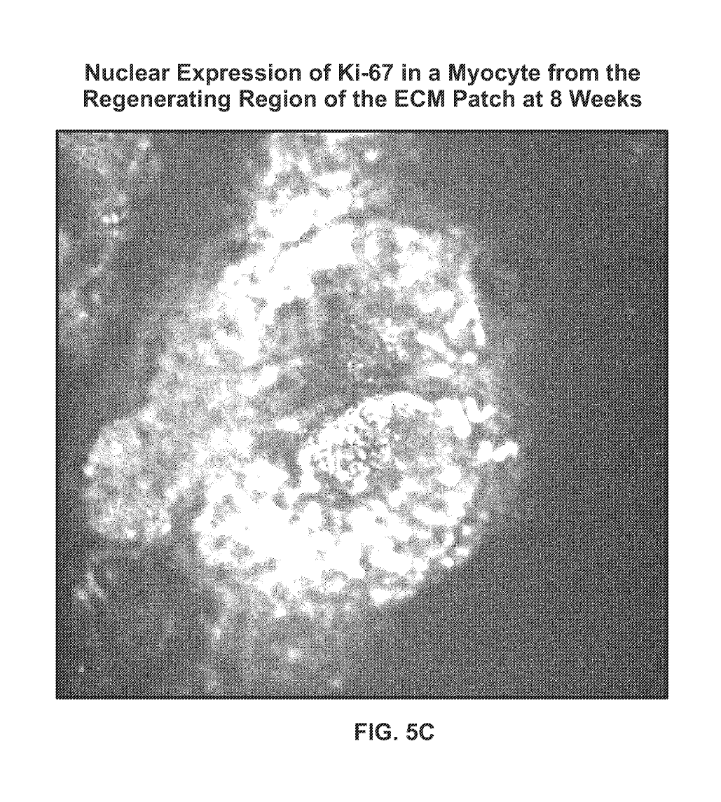

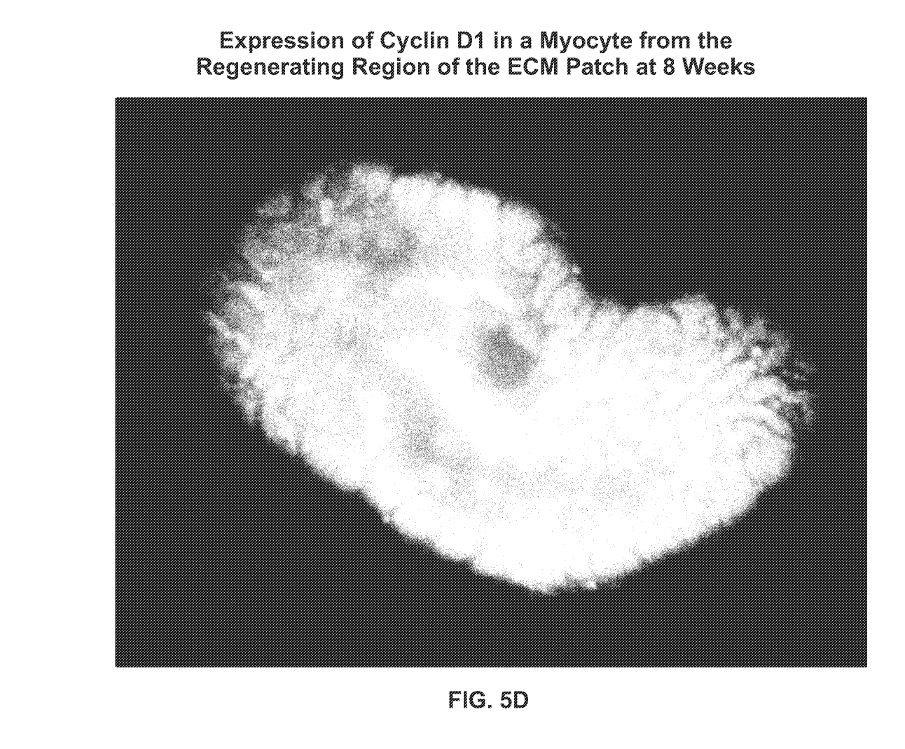

FIG. 5A demonstrates the presence of myocytes in the ECM implant region at eight weeks. FIG. 5B indicates Cyclin D1 expression was detected in the ECM implant region at eight weeks post-implantation. FIG. 5C demonstrates that nuclear expression of Ki-67 was observed in a myocyte from the regenerating region of the ECM patch at 8 weeks. Additionally, expression of CyclinD1 was observed in a myocyte from the regenerating region of the ECM patch at 8 weeks (FIG. 5D).

The experiments discussed above with ECM implants demonstrate that the lack of regeneration in the mammalian heart is likely related to an unfavourable environment, rather than an innate inability of the mammalian myocardium to regenerate. Replacement of myocardium with normal extracellular scaffold creates an environment that is favourable to myocardial regeneration. These favourable conditions are characterized by proliferation of c-kit+ stem cells that change the signalling pattern in the myocardium. Our in vitro model does not involve the use of ECM, thereby suggesting its importance in providing an environment for proliferating cells, rather than stimulating cells to proliferate. Our in vitro model further suggests that interaction of myocytes with stem cells can induce cardiomyocytes to enter the cell cycle, and it is this entry into the cell cycle that is likely to be an important part of stem cell assisted myocardium regeneration. In fact even conditioned media from the cultured hMSCs can induce myocyte proliferation (FIG. 4, panel H). This means that conditioned media independent of cells can in principle induce cardiac repair through myocyte proliferation.

### 7. EXAMPLE

### STEM CELL MEDIATED STIMULATION OF CARDIOMYOCYTE PROLIFERATION

The subsection below describes data demonstrating that implantation of a scaffold derived from bovine pericardium, i.e, Veritas®, is capable of stimulating proliferation of cardiomyocytes.

7.1. Materials and Methods

Human mesenchymal stem cells were obtained from BioWhittaker/Cambrex Inc. Cyclin-D1, Ki-67 and c-kit antibodies were purchased from Santa Cruz Biotechnology Inc. Antibody for α-sarcomeric actinin was purchased from Sigma. Urinary bladder extracellular scaffold membrane (ECM) was generously provided by Dr. Stephan Badylak (University of Pittsburgh). Veritas was obtained from Synovis Life Technologies.

To introduce an amputation wound in the canine heart a full thickness portion of myocardium of right ventricle approximately 15×10 mm was excised and replaced with membrane made of ECM, Dacron or Veritas® in adult mongrel dogs. All animal received humane care in accordance with the “Principles of Laboratory Animal Care” formulated by the National Society for Medical Research and the “Guide for the Care and Use of Laboratory Animals” of National Academy of Sciences (NIH publication No. 85-23) and treated according to protocol IACUC#20031326 approved by the Animal Care and Use Committee at SUNY Stony Brook,

7.2. Results

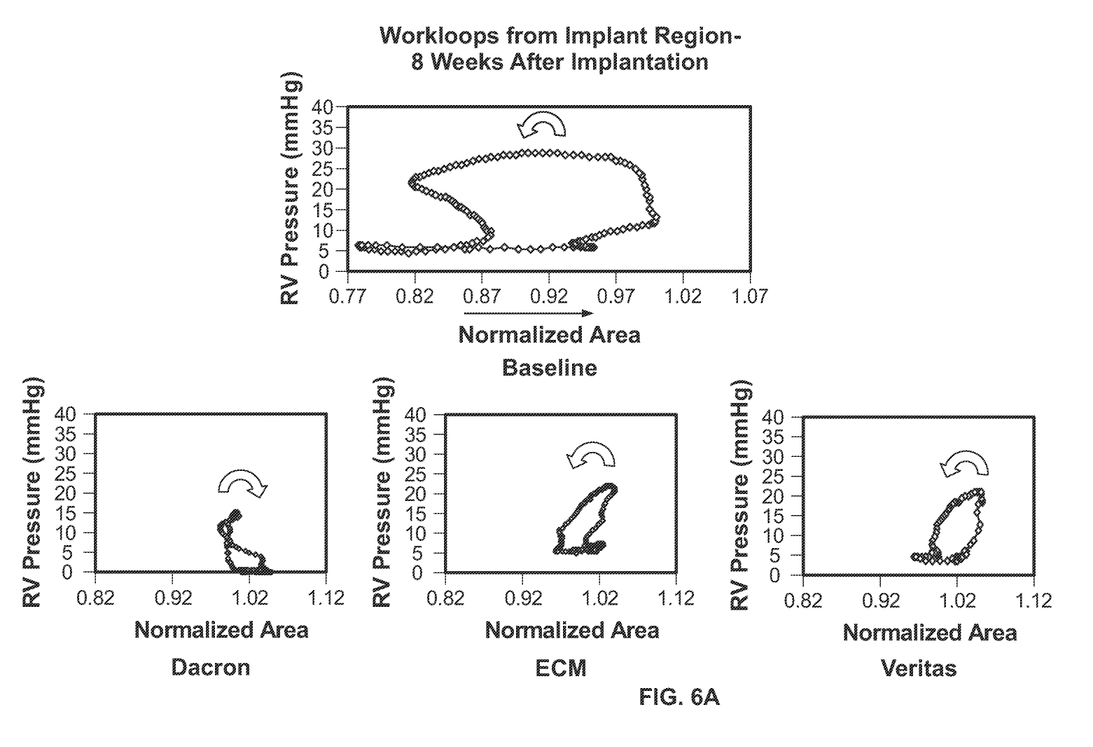

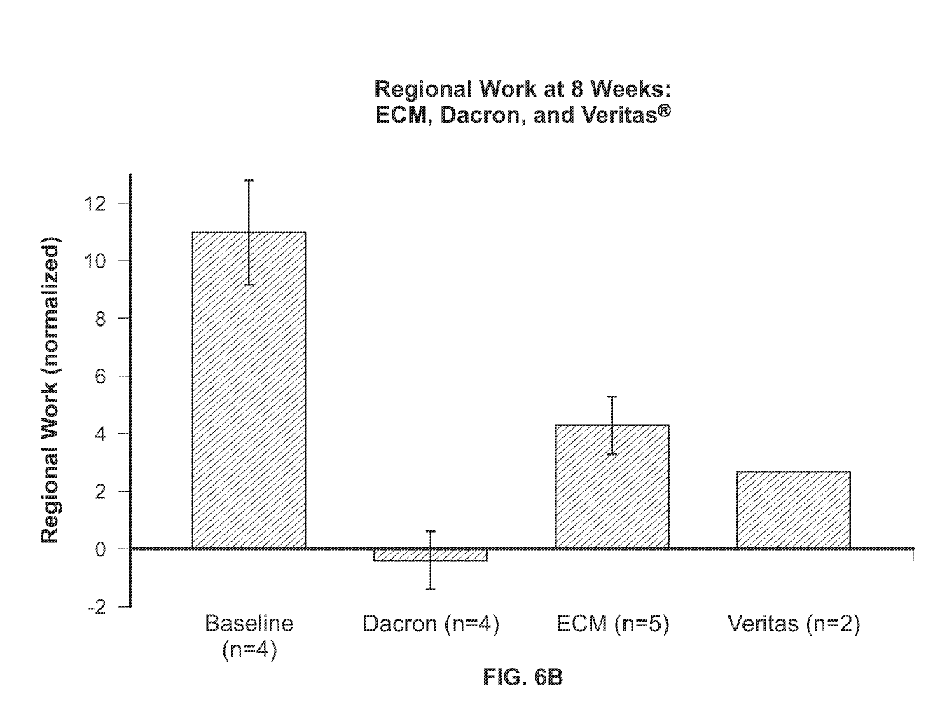

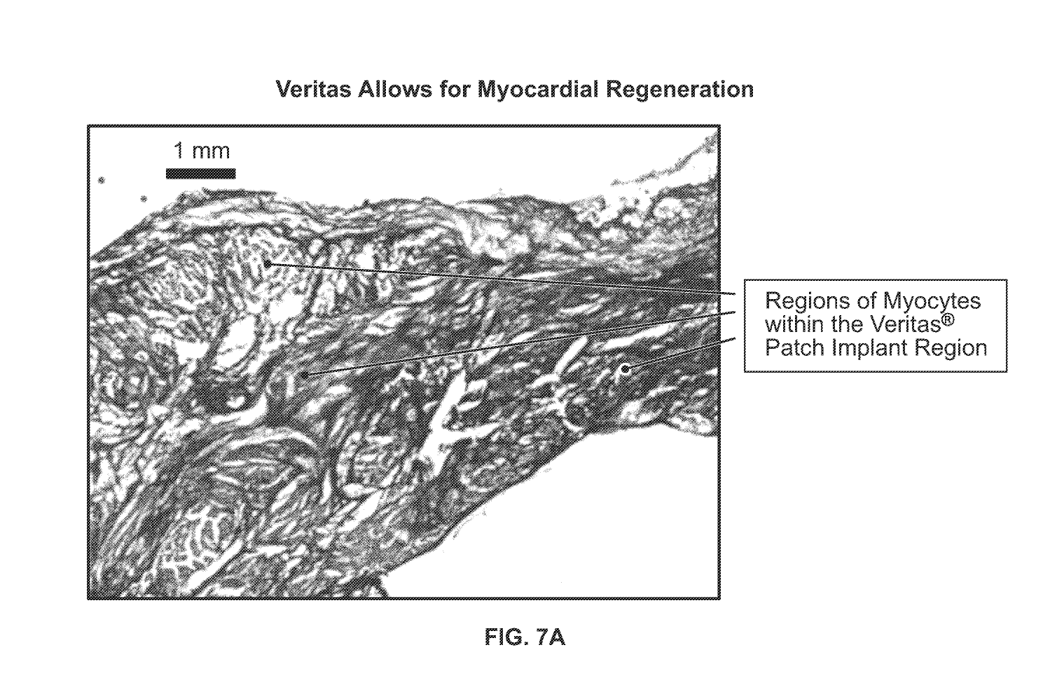

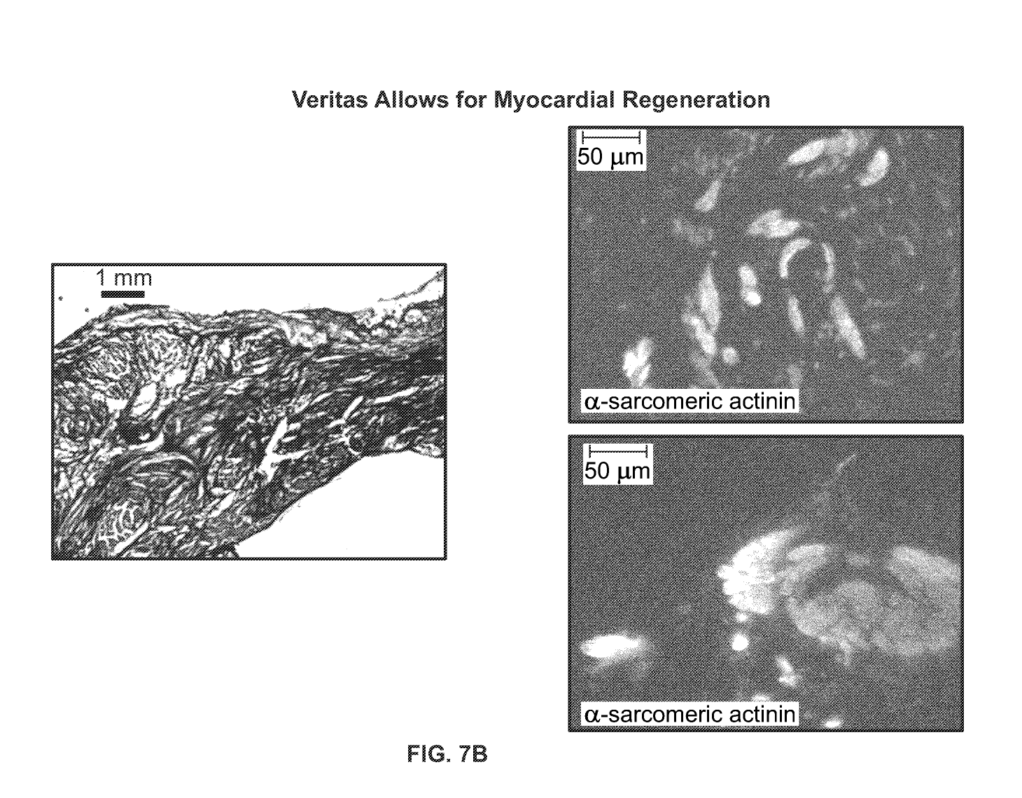

To investigate how generally healthy mammalian heart will respond to similar injuries a small (less than 5% of area) full thickness region of the canine right ventricle was excised and replaced with a patch made of either extracellular scaffold (ECM) prepared from swine bladder(5), Veritas® prepared from bovine pericardium or Dacron synthetic material. Regional heart performance was assayed eight weeks after implantation as previously described (6,7). Regional heart performance was assayed as previously described (Gaudette, G. R. et al., 2002, Cardiovascular Engineering: An International Journal 2, 129-137; Gaudette, G. R. et al., . . . , 2001, Ann. Biomed. Eng 29, 775-780 (2001). Hearts implanted with Veritas® demonstrated regional contractile properties similar to those obtained with ECM (FIG. 6A-B). The work loops generated by the implant region suggest the presence of cells contracting in sync with the native myocardium. Microscopic examination revealed that the myocardium was partially regenerated in the Veritas implant (FIG. 7A-B). Cells with myocyte morphology that stained positively for α-sarcomeric actinin (α-SA) were located in the Veritas implant.

### 8. EXAMPLE

### WNT-5A MEDIATED STIMULATION OF CARDIOMYOCYTE PROLIFERATION

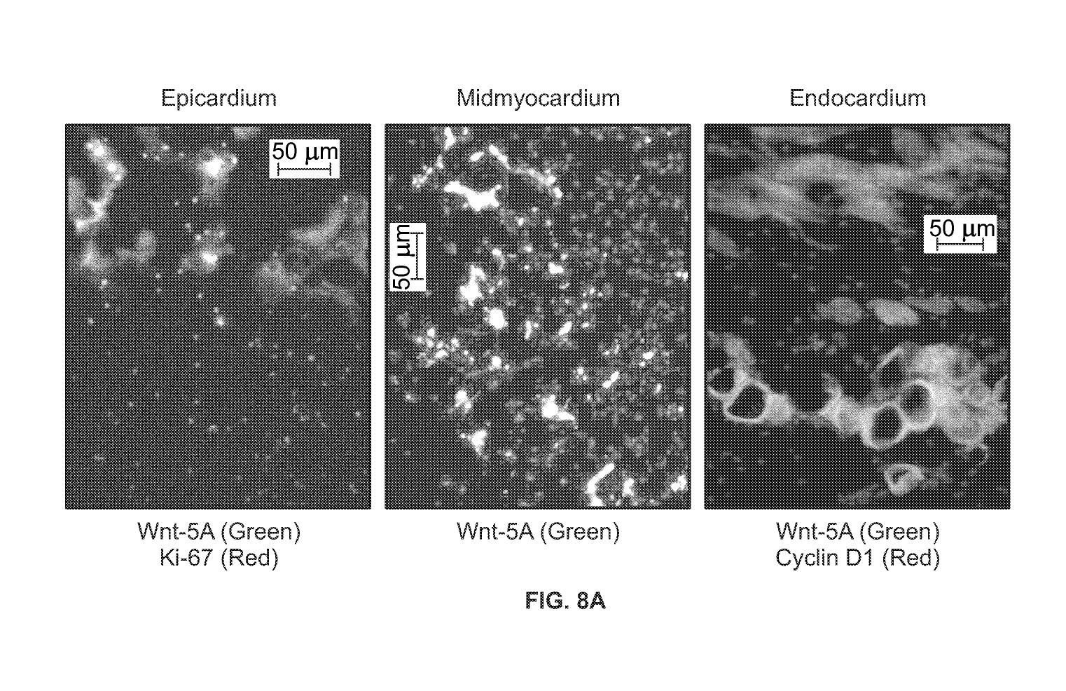

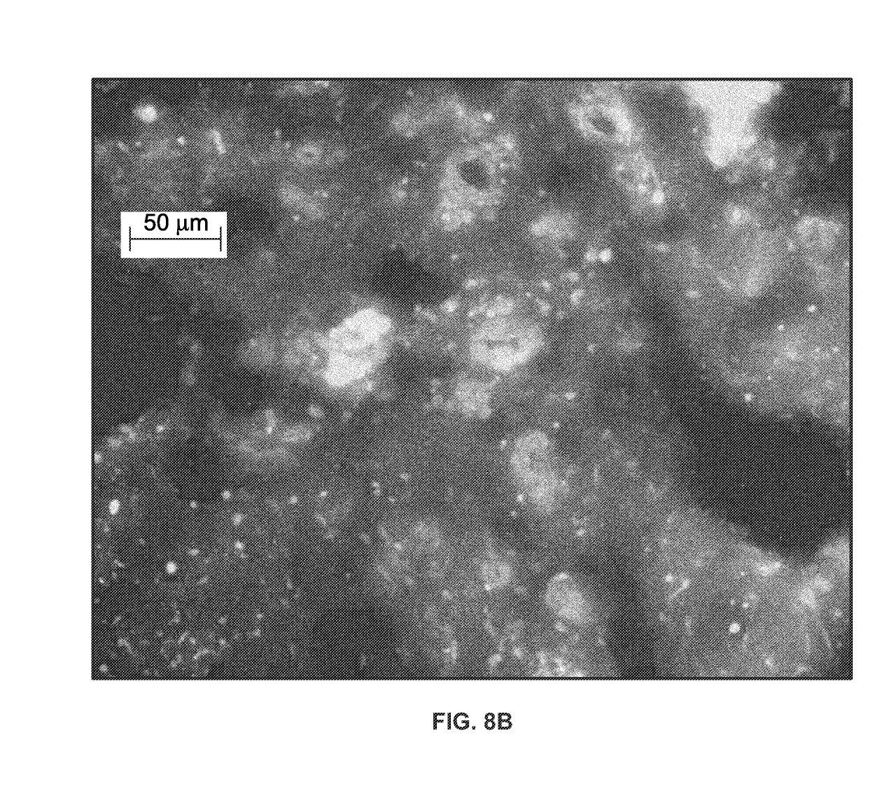

The subsection below describes data demonstrating that the Wnt-5A protein is capable of stimulating proliferation of cardiomyocytes. To detect Wnt-5A expression sections of 8 week implants were stained with Wnt-5A antibodies (R&D Systems). At this 8 week time point Wnt-5A expression correlates with mitotic propagation of myocytes. Wnt-5A was localized to epi- and endocardial surfaces of the implant as well as to new forming blood vessels (FIG. 8).

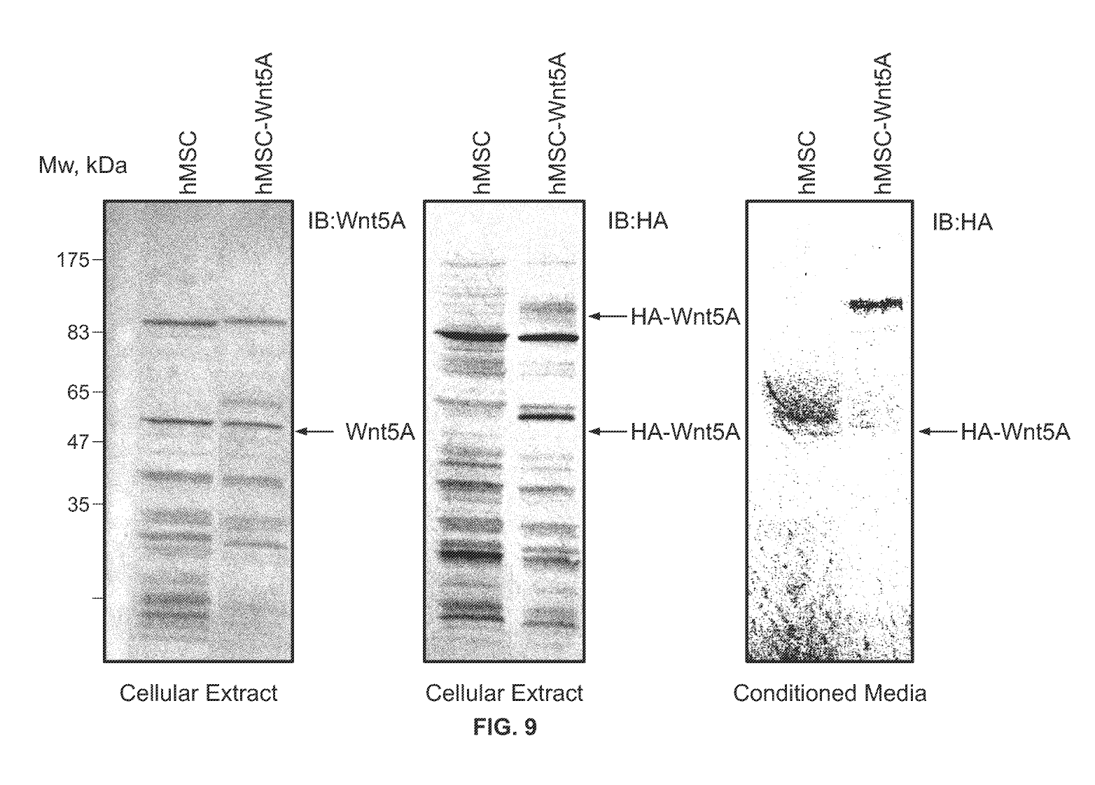

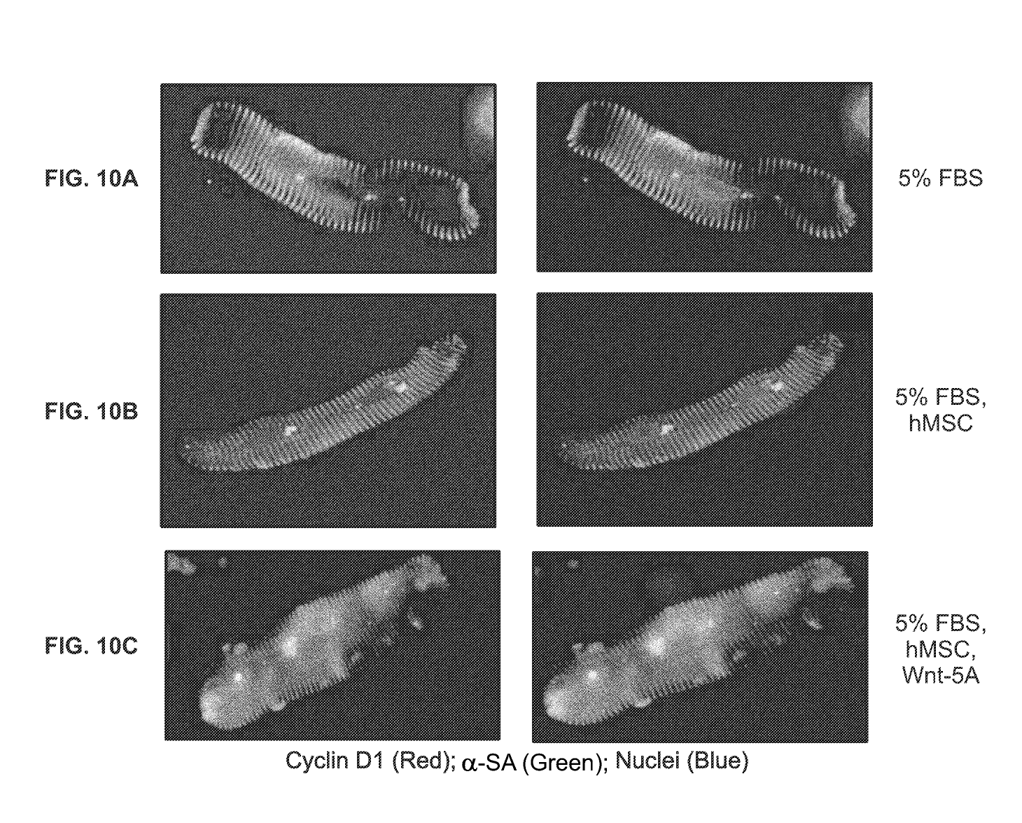

Wnt-5A was also overexpressed in human mesenchymal stem cells by electroporation (Nucleofector, Amaxa) of C-terminally tagged mouse Wnt-5A cloned in the pUSEamp mammalian expression vector (USTATE Biotechnology). Western Blot analysis demonstrated Wnt-5A overexpression in cellular lysates of human mesenchymal stem cells and in media conditioned by human mesenchymal stem cells (FIG. 9). Wnt-5A was detected both by antibodies for native Wnt-5A protein (R&D Systems) and antibodies against HA-Tag (Santa Cruz Biotechnology). To stimulate expression of cell cycle specific proteins in adult cardiac myocytes, myocytes were treated for four days with Dulbecco's Modified Eagle's Media containing 5% fetal bovine serum (DEM-FBS), or DEM-FBS conditioned by incubation with a confluent layer of human mesenchymal stem cells for two days, or DEM-FBS conditioned by incubation with a confluent layer of human mesenchymal stem cells overexpressing Wnt-5A protein for two days. FIG. 10 shows expression of cyclin D1 in cardiac myocytes after staining with polyclonal antibodies against cyclin D1 (Santa Cruz Biotechnology). Only those myocytes exposed to media conditioned by Wnt-5A overexpressing stem cells demonstrated cyclin D1 expression in the nuclei.

The present invention is not to be limited in scope by the specific embodiments described herein which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the claims. Throughout this application, various publications are referenced to by numbers. The disclosures of these publications in the entireties are hereby incorporated by reference into this application in order to more fully describe the state of the art to those skilled therein as of the date of the invention described and claimed herein.

We claim:

1. A method for inducing cardiomyocyte proliferation in a subject afflicted with a cardiac disorder, comprising administering an activator of the Wnt-5A mediated signal transduction pathway.

2. The method of claim 1 wherein the activator of the Wnt-5A mediated signal transduction pathway is a compound that activates a frizzled (fz) receptor.

3. The method of claim 2 wherein the frizzled (fz) receptor is selected from the group consisting of the frizzled-2, frizzled-3, frizzled-4, frizzled-5, frizzled-6, and frizzled-8 receptor.

4. The method of claim 1 wherein the activator of the Wnt-5A mediated signal transduction pathway is a Wnt-5A polypeptide.

5. The method of claim 1 wherein the activator of the Wnt-5A mediated signal transduction pathway is a Wnt-5A polypeptide fragment that induces cardiomyocyte proliferation.

6. The method of claim 1 wherein the activator of the Wnt-5A mediated signal transduction pathway is co-administered with at least one inducer of cardiomyocyte proliferation.

7. The method of claim 6 wherein the inducer of cardiomyocyte proliferation is selected from the group consisting of metalloproteases, platelet derived growth factor, brain derived neurotropic factor and insulin-like growth factor-1.

8. The method of claim 6 wherein the inducer of cardiomyocyte proliferation is insulin-like growth factor-1.

9. The method of claim 1 wherein the activator of the Wnt-5A mediated signal transduction pathway is a cell that expresses a Wnt-5A polypeptide.

10. The method of claim 9 wherein the cell is a human stem cell engineered to express the Wnt-5A polypeptide.

11. The method of claim 9 or 10 wherein the cell co-expresses insulin-like growth factor-1.

12. The method of claim 1 wherein the activator of the Wnt-5A mediated signal transduction pathway is a scaffold comprising a Wnt-5A polypeptide.

13. The method of claim 12 wherein the scaffold further comprises an inducer of cardiomyocyte proliferation selected from the group consisting of metalloproteases, platelet derived growth factor, brain derived neurotropic factor and insulin-like growth factor-1.

14. The method of claim 12 wherein the scaffold further comprises insulin-like growth factor-1.

15. The method of claim 1 wherein the activator of the Wnt-5A mediated signal transduction pathway is conditioned media derived from a cell that expresses a Wnt-5A polypeptide.

16. The method of claim 15 wherein the cell is a human stem cell engineered to express the Wnt-5A polypeptide.

17. The method of claim 1 wherein the activator of the Wnt-5A mediated signal transduction pathway comprises a Wnt-5A polypeptide and a physiologically acceptable carrier.

18. The method of claim 1 wherein the activator of the Wnt-5A mediated signal transduction pathway is a Wnt-5A polypeptide fragment that induces cardiomyocyte proliferation and a physiologically acceptable carrier.
